(19) United States
(12) Patent Application Publication
(10) Pub. No.: US 2004/0259085 A1
(43) Pub. Date: Dec. 23, 2004

(54) Methods and compositions for predicting prostate cancer

(76) Inventor: Chawnshang Chang, Pittsford, NY (US)

Correspondence Address: NEEDLE & ROSENBERG, P.C., SUITE 1000, 999 PEACHTREE STREET, ATLANTA, GA, 30309-3915, US

(21) Appl. No.: 10/333,894

(22) PCT Filed: Jul. 27, 2001
(86) PCT No.: PCT/US01/23834

Related U.S. Application Data
(60) Provisional application No. 60/221,074, filed on Jul. 27, 2000.

(51) Int. Cl. C12Q 1/68; C12P 19/34

(52) U.S. Cl. 435/6; 435/91.2

(57) ABSTRACT

Disclosed are compositions and methods for assessing risk in a subject for prostate cancer.

This application claims priority to U.S. Provisional Application No. 60/221,074 filed on Jul. 27, 2000, which application is herein incorporated by reference in its entirety.

### I. BACKGROUND OF THE INVENTION

The incidence of clinical prostate cancer differs substantially between ethnic groups, with African Americans having a 10- to 40-fold higher incidence than Asians (1-3AR). Such disparity in incidence of clinical prostate cancer cannot be explained entirely by population differences in screening. An earlier study shows that after adjustment for screening, there is still a 3- to 4-fold difference in incidence rates between U.S. and Japanese men, whose rates are among the highest in Asians (4AR). Despite the dramatic racial variation in clinical prostate cancer incidence, the prevalence of latent carcinoma appears to be similar across populations (5AR), suggesting that there exists differences in factors (either genetic or environmental) that promote the progression of microscopic tumors to clinically overt carcinoma.

The growth, differentiation, and proliferation of prostatic cells are regulated by androgens (6AR). The biological effects of androgens are mediated through binding to the intracellular androgen receptor (AR), which in turn regulates the transcription of target genes with the assistance of transcriptional coactivators (7AR).

II. SUMMARY OF THE INVENTION

In accordance with the purposes of this invention, as embodied and broadly described herein, this invention, in one aspect, relates compositions and methods for assessing prostate cancer risk.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### III. BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1. (AR) shows the percent distribution of the number of CAG repeats.

### IV. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

B. COMPOSITIONS

Disclosed are the components to be used to prepare the disclosed compositions as well as the compositions themselves and to be used within the methods disclosed herein. These and other materials are disclosed herein, and it is understood that when combinations, subsets, interactions, groups, etc. of these materials are disclosed that while specific reference of each various individual and collective permutation of these compounds may not be explicitly disclosed, each is specifically contemplated and described herein. For example, if a particular AIB1 protein or gene is disclosed and discussed and a number of modifications that can be made to a number of molecules including the AIB1 protein or gene are discussed, specifically contemplated is each and every combination and permutation of AIB1 protein or gene and the modifications that are possible unless specifically indicated to the contrary. Thus, if a class of molecules A, B, and C are disclosed as well as a class of molecules D, E, and F and an example of a combination molecule, A-D is disclosed, then even if it each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that one way to define any known variants and derivatives or those that might arise, of the disclosed genes and proteins herein is through defining the variants and derivatives in terms of homology to specific known sequences. For example, SEQ ID NO:18 sets forth a particular sequence of an AIB1 gene and SEQ ID NO:19 sets forth a particular sequence of the protein encoded by SEQ ID NO:18, an AIB1 protein. Specifically disclosed are variants of these and other genes and proteins herein disclosed which have at least, 70, 71, 72,73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, New York, 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least, 5, 10, 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least, 5, 10, 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least 5, 10, 15, 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

### 1. AIB1/SRC3

The AIB1 protein (Amplified In Breast cancer 1, also known as Steroid Receptor Coactivator-3, or SRC-3), encoded by the AIB1/SRC-3 gene located on chromosome 20 (20q12), is an AR coactivator and a member of the steroid receptor coactivator family, which interacts with members of the nuclear hormone receptor family (14,15 AIB1). Like the AR protein, the AIB1/SRC-3 coactivator contains a stretch of glutamine residues encoded by a variable-length track of CAG/CAA repeats in the AIB1/SRC-3 gene.

### a) Sequences

There are a variety of sequences related to the AIB1 gene having the following Genbank Accession Numbers: NT_{—}011362, XM_{—}030033, XM_{—}030032, XM_{—}009483, XM_{—}030031, XM_{—}030034, AL353777, AL034418, AL021394, AY008258, AF322224, NM_{—}008679, NM_{—}006534, AF012108, and AF044080, these sequences and others are herein incorporated by reference in their entireties as well as for individual subsequences contained therein.

One particular sequence set forth in SEQ ID NO:18 and having Genbank accession number XM_{—}030032 is used herein, as an example, to exemplify the disclosed compositions and methods. It is understood that the description related to this sequence is applicable to any sequence related to AIB1 unless specifically indicated otherwise. Those of skill in the art understand how to resolve sequence discrepancies and differences and to adjust the compositions and methods relating to a particular sequence to other related sequences (i.e. sequences of AIB1). Primers and/or probes can be designed for any AIB1 sequence given the information disclosed herein and known in the art.

### b) CAG/CAA region

The CAG/CAA region of the AIB1 gene is located in the coding region of the AIB 1 gene. For example, the CAG/CAA region in SEQ ID NO:18 can be defined by the region from nucleotide 3930 to nucleotide 4016. In certain embodiments of the disclosed compositions and methods, this represents the CAG/CAA region of the AIB1 gene. However, it is understood that various mutations, alterations, or other genetic variation including allelic variation can occur in certain individuals and those of skill in the art understand how to locate this region within any given AIB1 gene variant. Thus, for example, if a nucleic acid is amplified from SEQ ID NO:18 or any variant of the AIB1 gene, that contains only only a fragment of the AIB1 gene, for example, a fragment of 1000 nucleotides, it is understood that the CAG/CAA region could be located within this molecule if it is included in the molecule, in whole or in part.

### c) Primers and Probes

Disclosed are compositions including primers and probes, which are capable of interacting with the AIB1 gene as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the AIB1 gene or region of the AIB1 gene or they hybridize with the complement of the AIB1 gene or complement of a region of the AIB1 gene.

Disclosed are primers that are capable of amplifying the CAG/CAA region of the AIB1 gene. In certain embodiments the primers amplify the CAG/CAA region of the AIB1 gene from nucleotide 3930 to nucleotide 4016 of the sequence set forth in SEQ ID NO:18. In certain embodiments the primers are “outside” the CAG/CAA region of the AIB1 gene. By outside the region it is intended to indicate that no region of the primer is intended to interact directly with the CAG/CAA region. For example, a primer outside the CAG/CAA region of the sequence set forth in SEQ ID NO:18 could hybridize with nucleotide 4017 to nucleotide 4037 of SEQ ID NO:18, but it would not be intended to hybridize with nucleotide 4016, and under the conditions designed for the enzymatic manipulation of the primer, it would not appreciably interact with nucleotide 4016. In other embodiments the primers are designed to interact with one or more nucleotides considered to be part of the CAG/CAA region, which primers herein are referred to as “inside” primers. Thus, an inside primer can include a primer region that can under conditions appropriate for the desired enzymatic manipulation hybridize with one or more nucleotides considered within the CAG/CAA region and contain a region that hybridizes with nucleotides that are not considered part of the CAG/CAA region. Thus, in one embodiment an inside primer would interact with the nucleotide at position 4016 of SEQ ID NO:18.

The size of the primers for interaction with the AIB1 gene in certain embodiments can be any size that supports the desired enzymatic manipulation of the primer, such as DNA amplification. A typical AIB1 primer would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments an AIB1 primer can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In certain embodiments the primers are designed such that they are outside primers whose nearest point of interaction with the AIB1 gene is within 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, or 200 nucleotides of the outermost defining nucleotide of the CAG/CAA region or complement of the CAG/CAA region.

In certain embodiments the primers are designed such that they are outside primers whose nearest point of interaction with the AIB1 gene is at least 0, 1, 2, 3, 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, or 200 nucleotides away from the outermost defining nucleotide of the CAG/CAA region or complement of the CAG/CAA region.

For example, with respect to the AIB1 gene set forth in SEQ ID NO:18, certain embodiments of the primers would be designed such that they are outside primers whose nearest point of interaction with the AIB1 gene occurs at position 4017, 4018, 4019, 4020, 4021, 4022, 4023, 4024, 4025, 4026, 4027, 4028, 4029, 4030, 4031, 4032, 4033, 4034, 4035, 4036, 4037, 4038, 4039, 4040, 4041, 4042, 4043, 4044, 4045, 4046, 4047, 4048, 4049, 4050, 4051, 4052, 4053, 4054, 4055, 4056, 4057, 4058, 4059, 4060, 4061, 4062, 4063, 4064, 4065, 4066, 4067, 4068, 4069, 4070, 4071, 4072, 4073, 4074, 4075, 4076, 4077, 4078, 4079, 4080, 4081, 4082, 4083, 4084, 4085, 4086, 4087, 4088, 4089, 4090, 4091, 4092, 4093, 4094, 4095, 4096, 4097, 4098, 4099, 4100, 4101, 4102, 4103, 4104, 4105, 4106, 4107, 4108, 4109, 4110, 4111, 4112, 4113, 4114, 4115, 4116, 4117, 4142, 4167, 4192, 4217 of SEQ ID NO:18.

The primers for the AIB1 gene typically will be used to produce an amplified DNA product that contains the CAG/CAA region of the AIB1 gene. In general, typically the size of the product will be such that the size can be accurately determined to within 3, or 2 or 1 nucleotides.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

### 2. Androgen Receptor

Androgen receptor (AR) plays a key role in intraprostatic androgenic action. Within the prostate gland, testosterone is converted into dihydrotestosterone (DHT), a more potent androgen. DHT then binds to the AR to form an intracellular DHT-AR complex, which in turn modulates prostatic target genes to induce proliferation.

The AR protein, consisting of 918 amino acids and encoded singly by the AR gene located on the X chromosome (Xq11-12), has three major functional domains: a transactivating amino-terminal domain, a DNA binding domain, and a ligand (steroid) binding domain (8AR). The open reading frame of the AR gene is separated over eight exons and has a length of 2,730 base pair (bp). The sequence encoding the large amino-terminal transactivating domain is found in the first exon; the DNA binding domain is encoded by exons 2 and 3; and the information for the ligand binding domain is distributed over exons 4 to 8 (8AR).

The first exon of the AR gene contains two polymorphic trinucleotide repeat segments that encode polyglutamine and polyglycine tracts localized in the N-terminal transactivation domain of the AR protein. The polyglutamine tract is encoded by a CAG trinucleotide repeat, and the polyglycine stretch by a GGN repeat. The number of CAG repeats ranges from about 8 to 35 repeats in normal individuals. Longer CAG repeat lengths appear to result in reduced AR transcriptional activity both in vivo and in vitro (9,10AR). Otherwise healthy men whose androgen receptor has a CAG repeat length at the long end of the normal range (>28) have an increased incidence of impaired spermatogenesis and infertility (11AR), conditions that are extremely androgen-dependent (12AR). Expansion of the CAG repeat length to over 40 repeats is related to a rare neuromuscular disorder, spinal and bulbar muscular atrophy (Kennedy syndrome), which is also associated with androgen insensitivity, decreased virilization, testicular atrophy, reduced sperm production, and infertility (13-15AR). Together, these clinical data suggest that a longer CAG repeat length decreases the functional competence of AR.

The length of the polyglycine (GGN) tract varies from about 10 to 30 repeats. The functional consequences of variation in the GGN tract are less clear. Deletion of the polyglycine tract reduces AR transcriptional activity by about 30% in transient transfection assays (16AR), although there is no significant correlation between polyglycine tract length and infertility (11AR).

### a) Sequences

There are a variety of sequences related to the androgen receptor gene having the following Genbank Accession Numbers: NM_{—}000044 or L49399 are two examples. These sequences and others available on Genbank are herein incorporated by reference in their entireties as well as for individual subsequences contained therein.

One particular sequence set forth in SEQ ID NO:20 and having Genbank accession number NM_{—}000044 is used herein, as an example, to exemplify the disclosed compositions and methods. It is understood that the description related to this sequence is applicable to any sequence related to androgen receptor unless specifically indicated otherwise. Those of skill in the art understand how to resolve sequence discrepancies and differences and to adjust the compositions and methods relating to a particular sequence to other related sequences. Primers and/or probes can be designed for any androgen receptor sequence given the information disclosed herein and known in the art.

### b) CAG/CAA Region

The CAG/CAA region in SEQ ID NO:20 can be defined by the region from nucleotide 1286 to nucleotide 1348. In certain embodiments of the disclosed compositions and methods, this represents the CAG/CAA region of the androgen receptor gene. However, it is understood that various mutations, alterations, or other genetic variation including allelic variation can occur in certain individuals and those of skill in the art understand how to locate this region within any given androgen receptor gene variant. Thus, for example, if a nucleic acid is amplified from SEQ ID NO:20 or any variant of the androgen receptor gene, that contains only 1000 nucleotides, it is understood that the CAG/CAA region could be located within this molecule if it is included in the molecule, in whole or in part.

### c) GGN Region

The GGN region in SEQ ID NO:20 can be defined by the region from nucleotide 2459 to nucleotide 2530. In certain embodiments of the disclosed compositions and methods, this represents the GGN region of the androgen receptor gene. However, it is understood that various mutations, alterations, or other genetic variation including allelic variation can occur in certain individuals and those of skill in the art understand how to locate this region within any given androgen receptor gene variant. Thus, for example, if a nucleic acid is amplified from SEQ ID NO:20 or any variant of the androgen receptor gene, that contains only 1000 nucleotides, it is understood that the GGN region could be located within this molecule if it is included in the molecule, in whole or in part.

### d) Primers and Probes

Disclosed are compositions including primers and probes, which are capable of interacting with the androgen receptor gene as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the androgen receptor gene or region of the androgen receptor gene or they hybridize with the complement of the androgen receptor gene or complement of a region of the androgen receptor gene.

Disclosed are primers that are capable of amplifying the CAG/CAA and/or GGN region of the androgen receptor gene. In certain embodiments the primers amplify the CAG/CAA region of the androgen receptor gene from nucleotide 1286 to nucleotide 1348 of the sequence set forth in SEQ ID NO:20. In certain embodiments the primers are “outside” the CAG/CAA region of the androgen receptor gene. By outside the region it is intended to indicate that no region of the primer is intended to interact directly with the CAG/CAA region. For example, a primer outside the CAG/CAA region of the sequence set forth in SEQ ID NO:20 could hybridize with nucleotide 1349 to nucleotide 1369 of SEQ ID NO:20, but it would not be intended to hybridize with nucleotide 1348, and under the conditions designed for the enzymatic manipulation of the primer, it would not appreciably interact with nucleotide 1348. In other embodiments the primers are designed to interact with one or more nucleotides considered to be part of the CAG/CAA region, which primers herein are referred to as “inside” primers. Thus, an inside primer can include a primer region that can under conditions appropriate for the desired enzymatic manipulation hybridize with one or more nucleotides considered within the CAG/CAA region and contain a region that hybridizes with nucleotides that are not considered part of the CAG/CAA region. Thus, in one embodiment an inside primer would interact with the nucleotide at position 1348 of SEQ ID NO:20.

In certain embodiments the primers amplify the GGN region of the androgen receptor gene from nucleotide 2459 to nucleotide 2530 of the sequence set forth in SEQ ID NO:20. In certain embodiments the primers are “outside” the GGN region of the androgen receptor gene. By outside the region it is intended to indicate that no region of the primer is intended to interact directly with the GGN region. For example, a primer outside the GGN region of the sequence set forth in SEQ ID NO:20 could hybridize with nucleotide 2531 to nucleotide 2551 of SEQ ID NO:20, but it would not be intended to hybridize with nucleotide 2530, and under the conditions designed for the enzymatic manipulation of the primer, it would not appreciably interact with nucleotide 2530. In other embodiments the primers are designed to interact with one or more nucleotides considered to be part of the GGN region, which primers herein are referred to as “inside” primers. Thus, an inside primer can include a primer region that can under conditions appropriate for the desired enzymatic manipulation hybridize with one or more nucleotides considered within the GGN region and contain a region that hybridizes with nucleotides that are not considered part of the GGN region. Thus, in one embodiment an inside primer would interact with the nucleotide at position 2530 of SEQ ID NO:20.

The size of the primers for interaction with the androgen receptor gene in certain embodiments can be any size that supports the desired enzymatic manipulation of the primer, such as DNA amplification. A typical androgen receptor primer would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments an androgen receptor primer can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In certain embodiments the primers are designed such that they are outside primers whose nearest point of interaction with the androgen receptor gene is within 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, or 200 nucleotides of the outermost defining nucleotide of the CAG/CAA and/or GGN region or complement of the CAG/CAA and/or GGN region.

In certain embodiments the primers are designed such that they are outside primers whose nearest point of interaction with the androgen receptor gene is at least 0, 1, 2, 3, 4, 5, 6, 7, 8, 9, 10, 11, 12 13,14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, or 200 nucleotides away from the outermost defining nucleotide of the CAG/CAA and/or GGN region or complement of the CAG/CAA and/or GGN region.

For example, with respect to the androgen receptor gene set forth in SEQ ID NO:20, certain embodiments of the primers would be designed such that they are outside primers whose nearest point of interaction with the androgen receptor gene occurs at position 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444,1445, 1446, 1447, 1448, 1449, 1474, 1499, 1524, or 1549 of SEQ ID NO:20.

The primers for the androgen receptor gene typically will be used to produce an amplified DNA product that contains the CAG/CAA and/or GGN region of the androgen receptor gene. In general, typically the size of the product will be such that the size can be accurately determined to within 3, or 2 or 1 nucleotides.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

### 3. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit for assessing a subject's risk for acquiring prostate cancer, comprising the oligonucleotides set forth in SEQ ID Nos: 1 and 2.

### C. METHODS OF MAKING THE COMPOSITIONS

The compositions disclosed herein and the compositions necessary to perform the disclosed methods can be made using any method known to those of skill in the art for that particular reagent or compound unless otherwise specifically noted. For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

### D. METHODS OF USING THE COMPOSITIONS

It is understood that any variation or implementation of the compositions discussed herein is understood to be a particular embodiment which can be used and is described as being used in the disclosed methods. Disclosed are methods for assessing the risk that a subject may acquire prostate cancer. Methods are also disclosed for assessing the clinical significance of the prostate cancer that a subject will get or already has. These methods involve assaying the length of regions of genes that have been shown herein to be related to the onset and severity of prostate cancer. The methods involve assaying regions of both the AIB1 gene and the AR gene either individually or collectively. There are particular regions of the AIB1 gene and the AR gene which have been shown to be related to the onset and severity of prostate cancer and these regions are found in all forms and variants of these genes. In one embodiment of the disclosed methods the length of the CAG/CAA repeat in either the AIB1 gene or AR gene of a subject, discussed above, is determined. This length can then be compared to known lengths, which have been shown to be correlated with the onset and severity of prostate cancer. For example, in the AIB1 gene, the CAG/CAA region determined to be greater than, less than, or equal to 29 repeats. When the number of repeats present in the repeat region of the subject is less than or more than 29 at least one allele of the subject's AIM1 gene, this person is determined to have an increased risk of developing prostate cancer. It is understood that since the AIB1 gene is present on chromosome 20, subjects will have two alleles of this gene. When a single allele has a length different than 29 the subject has an increased risk of acquiring prostate cancer. If, however, the subject has two alleles of the AIB1 gene that both have lengths in the CAG/CAA repeat region different than 29, the subject has a higher risk of acquiring prostate cancer than if only one allele has a CAG/CAA length different than 29. Thus, if the alleles of the AIB1 gene are assayed individually, if the first allele assayed has a CAG/CAA repeat length different than 29, a certain amount of information as to the person's susceptibility will be gained, however, if the allele contains a CAG/CAA repeat equal to 29, the second allele must be looked at to get meaningful information as to the person's likelihood of acquiring prostate cancer. Furthermore, more information will be gained by assaying the second allele even if the first allele has a CAG/CAA length less than or greater than 29 because the second allele may also have a CAG/CAA length different 29 and this provides more information about the subject's prostate cancer susceptibility. Thus, it is preferred that both alleles of the AIB1 gene are assayed when performing the methods herein.

The CAG/CAA region of the subject's AR gene can also be assayed. However, in the AR length the length of the subject's CAG/CAA region in the AR gene is compared to 23 repeats. If there are more or less than 23 repeats the subject has an increased risk of prostate cancer. Furthermore, the risk of prostate cancer in a subject is directly correlated with the length of the subject's CAG/CAA repeat. The greater the deviation of the number of CAG/CAA repeats in a subjects DNA from 23 repeats the greater the likelihood that the person will acquire prostate cancer.

### 1. Methods Assaying the CAG/CAA Repeat of the AIB1 Gene

Methods are disclosed where only the length of CAG/CAA repeat in a subject's AIB1 gene is assayed. Disclosed are methods for assessing the risk of developing prostate cancer in a human by analyzing the AIB1 gene of the subject.

Disclosed are methods for assessing the risk of prostate cancer in a human subject comprising determining the length of the contiguous CAG or CAA repeats in both AIB1 gene alleles of the subject and assessing whether the length of the CAG or CAA repeats is less than, equal to, or greater than 29 repeats, a length less than or greater than 29 repeats in both alleles indicating an increased risk of prostate cancer in the subject.

Also disclosed are methods where determining the length of the repeats comprises amplifying a region of both AIB1 gene alleles comprising the contiguous CAG or CAA repeat. Any type of amplification method can be employed to amplify the target regions. Preferred methods include PCR and direct sequencing of the target repeat regions.

When two repeat regions of two alleles are amplified two products will be produced. These products can then be assayed for their length, by for example, gel chromotagraphy, HPLC, or capillary gel electrophoresis. Thus, disclosed are methods that produce two PCR products and methods where the PCR products are analyzed by chromatography, including but not limited to gel electrophoresis.

In certain embodiments the sequence of the repeat region can be determined by, for example, direct sequencing of the repeat region. In some embodiments the PCT product or other DNA amplification product, can itself be sequenced.

In certain embodiments of the disclosed methods, the repeat region is assayed or amplified by targeting primers or probes to the region or the areas of the target gene surrounding the region. These primers, for example, can be used to amplify the region by for example, PCR. It is preferred that the method utilize primers as they are discussed herein. For example, the PCR product can be produced using a first AIB1 primer that selectively hybridizes with the complement of sequence 5′ to the repeat region and a second AIB1 primer that selectively hybridizes with sequence 3′ to the repeat region.

In certain embodiments, the PCR product can be produced using a first AIB1 primer that selectively hybridizes with the sequence set forth in SEQ ID NO:3 and a second AIB1 primer that selectively hybridizes with the sequence set forth in SEQ ID NO:4. In still other embodiments of the disclosed methods, the first AIB1 primer has the sequence set forth in SEQ ID NO:1 and the second AIB1 primer has the sequence set forth in SEQ ID NO:2.

In certain embodiments determining how many CAG or CAA repeats there are comprises sequencing the CAG or CAA repeats.

### 2. Methods Assaying the CAG/CAA Repeat of the Androgen Receptor Gene

Just as with the AIB1 gene, there are repeat regions within the androgen receptor gene that can be assayed and used to assess a person's risk for acquiring prostate cancer. There are two repeat regions that can be assayed within the androgen receptor gene. The CAG/CAA repeat region within the AR gene can be assayed as discussed for the CAG/CAA repeat region of the AIB1 gene. There is a key difference, however, and that is the length of the subject's CAG/CAA repeat region in the AR gene is compared to 23 repeats, not 29 repeats. The predictability of acquiring prostate cancer arises from a comparison to 23 CAG/CAA repeats. Furthermore, the CAG/CAA repeat with in the AR gene is predictive based on single repeat changes. Thus, the likelihood of acquiring prostate cancer increases for each difference in length from 23 repeats. Thus, for example, a subject having 15 repeats is more likely to acquire prostate cancer or have a clinically significant prostate cancer than a person having 16 repeats and a subject having 16 repeats is more likely to acquire prostate cancer or have a clinically significant prostate cancer than a person having 17 repeats and a subject having 17 repeats is more likely to acquire prostate cancer or have a clinically significant prostate cancer than a person having 16 repeats and so forth. This relationship holds for all lengths of CAG/CAA repeats in the AR gene.

The AR gene also has a GGN repeat region whose length is related to the susceptibility of prostate cancer. The methods and reagents disclosed for the CAG/CAA repeats of the AR gene apply to the GGN repeat within the AR gene and are all specifically contemplated herein.

### 3. Methods Assaying the CAG/CAA Repeat of the Androgen Receptor Gene and the AIB1 Gene

Methods are also disclosed where both the AIB1 gene and the AR gene are assayed to arrive at a likelihood that a subject will acquire prostate cancer. It is understood that all of the conditions and permutations of performing the methods that involve assaying either AIB1 or AR alone can also be used and applied when both AIB1 and AR are assayed.

Disclosed are methods for assessing the risk of developing prostate cancer in a human by analyzing the AIB1 gene and the AR gene of the subject together.

For example, disclosed are methods for assessing the risk of prostate cancer in a human subject comprising determining the length of the contiguous CAG or CAA repeats in the AIB1 gene alleles of the subject and assessing whether the length of the CAG or CAA repeats in each allele is less than, equal to, or greater than 29 repeats, and determining the length of the contiguous CAG or CAA repeats in the androgen receptor gene of the subject and assessing whether the length of the CAG or CAA repeats is less than, equal to, or greater than 23 repeats, a length in at least one allele less than or greater than 29 repeats in the AIB1 gene and less than or greater than 23 repeats in the androgen receptor gene indicating an increased risk of prostate cancer in the subject.

Also disclosed are methods wherein determining the length of the contiguous CAG or CAA repeats in the AIB1 gene alleles comprises amplifying a region of the AIB1 gene alleles comprising the CAG or CAA repeats and wherein determining the length of the CAG or CAA repeats in the androgen receptor gene comprises amplifying a region of the androgen receptor gene comprising the CAG or CAA repeats.

Furthermore, methods are disclosed wherein the amplification of the regions of the AIB1 gene and the androgen receptor gene is by PCR that produces a first and a second AIB1 PCR product and an androgen receptor PCR product.

Disclosed are methods further comprising analyzing the PCR products by chromatography and methods wherein the chromatography is gel electrophoresis. The sequence of the PCR products can be determined.

The methods involving amplifying repeat regions in both AIB1 and the AR genes can for example involve primers. It is understood that primers specific for each repeat region would be used. For example, the AIB1 PCR product is produced using a first AIB1 primer that selectively hybridizes with the sequence set forth in SEQ ID NO:3 and a second AIB1 primer that selectively hybridizes with the sequence set forth in SEQ ID NO:4. Additional methods are wherein the first AIB1 primer has the sequence set forth in SEQ ID NO:1 and the second AIB1 primer has the sequence set forth in SEQ ID NO:2.

Or when assaying both genes the androgen receptor PCR product is produced using a first androgen receptor CAG primer that selectively hybridizes with the sequence set forth in SEQ ID NO:.9 and a second androgen receptor CAG primer that selectively hybridizes with the sequence set forth in SEQ ID NO:10. In some embodiments the first androgen receptor CAG primer has the sequence set forth in SEQ ID NO:7 and the second androgen receptor CAG primer has the sequence set forth in SEQ ID NO:8.

Or when both the AIB1 and the AR genes are assayed, the AIB1 PCR product is produced using a first AIB1 primer that selectively hybridizes with the sequence set forth in SEQ ID NO:3 and a second AIB1 primer that selectively hybridizes with the sequence set forth in SEQ ID NO:4. and wherein the androgen receptor PCR product is produced using a first androgen receptor CAG primer that selectively hybridizes with the sequence set forth in SEQ ID NO:9 and a second androgen receptor CAG primer that selectively hybridizes with the sequence set forth in SEQ ID NO:10.

In certain embodiments the first AIB1 primer has the sequence set forth in SEQ ID NO:1 and the second AIB1 primer has the sequence set forth in SEQ ID NO:2 and wherein the first androgen receptor CAG primer has the sequence set forth in SEQ ID NO:7 and the second androgen receptor CAG primer has the sequence set forth in SEQ ID NO:8.

In some embodiments the methods are performed wherein more than 29 contiguous CAG or CAA repeats in at least one allele of the AIB1 gene of the person and more than 23 contiguous CAG or CAA repeats in the androgen receptor gene of the person indicates an increased risk of prostate cancer in the subject.

In other embodiments the methods are performed wherein more than 29 contiguous CAG or CAA repeats in at least one allele of the AIB1 gene of the person and less than 23 contiguous CAG or CAA repeats in the androgen receptor gene of the person indicates an increased risk of prostate cancer in the subject.

In some embodiments the methods are performed wherein less than 29 contiguous CAG or CAA repeats in at least one allele of the AIB1 gene of the person and more than 23 contiguous CAG or CAA repeats in the androgen receptor gene of the person indicates an increased risk of prostate cancer in the subject.

In other embodiments the methods are performed wherein less than 29 contiguous CAG or CAA repeats in at least one allele of the AIB1 gene of the person and less than 23 contiguous CAG or CAA repeats in the androgen receptor gene of the person indicates an increased risk of prostate cancer in the subject.

In other embodiments the methods are performed wherein more or less than 29 contiguous CAG or CAA repeats in both alleles of the AIB1 gene of the person and more than 23 contiguous CAG or CAA repeats in the androgen receptor gene of the person indicates an increased risk of prostate cancer in the subject.

In other embodiments the methods are performed wherein more or less than 29 contiguous CAG or CAA repeats in both alleles of the AIB1 gene of the person and less than 23 contiguous CAG or CAA repeats in the androgen receptor gene of the person indicates an increased risk of prostate cancer in the subject.

In other embodiments the methods are performed wherein more than 29 contiguous CAG or CAA repeats in both alleles of the AIB1 gene of the person and more than 23 contiguous CAG or CAA repeats in the androgen receptor gene of the person indicates an increased risk of prostate cancer in the subject.

In other embodiments the methods are performed wherein less than 29 contiguous CAG or CAA repeats in both alleles of the AIB1 gene of the person and less than 23 contiguous CAG or CAA repeats in the androgen receptor gene of the person indicates an increased risk of prostate cancer in the subject.

Also disclosed are methods for assessing the risk of prostate cancer in a human subject comprising determining the length of the contiguous CAG or CAA repeats in the AIB1 gene alleles of the subject and assessing whether the length of the CAG or CAA repeats in each allele is less than, equal to, or greater than 29 repeats, and determining the length of the contiguous GGN repeats in the androgen receptor gene of the subject and assessing whether the length of the GGN repeats is less than, equal to, or greater than 23 repeats, a length in at least one allele less than or greater than 29 repeats in the AIB1 gene and less than or greater than 23 repeats in the androgen receptor gene indicating an increased risk of prostate cancer in the subject, wherein N is either T, G, or C.

Disclosed are methods wherein determining the length of the contiguous CAG or CAA repeats in the AIB1 gene alleles comprises amplifying a region of the AIB1 gene alleles comprising the CAG or CAA repeats and wherein determining the length of the contiguous GGN repeats in the androgen receptor gene comprises amplifying a region of the androgen receptor gene comprising the GGN repeats.

Further methods are disclosed wherein the amplification of the regions of the AIB1 gene and the androgen receptor gene is by PCR that produces a first and a second AIB1 PCR product and an androgen receptor PCR product.

In other methods the amplification further comprises analyzing the PCR products by chromatography.

The amplification products including the PCR products can be analyzed by gel electrophoresis.

In other embodiments the sequence of the PCR products is determined.

Disclosed are amplification methods wherein the AIB1 PCR product is produced using a first AIB1 primer that selectively hybridizes with the sequence set forth in SEQ ID NO:3 and a second AIB1 primer that selectively hybridizes with the sequence set forth in SEQ ID NO:4.

Also disclosed are amplification methods wherein the first AIB1 primer has the sequence set forth in SEQ ID NO: 1 and the second AIB1 primer has the sequence set forth in SEQ ID NO:2.

Disclosed are methods wherein the androgen receptor PCR product is produced using a first androgen receptor GGN primer that selectively hybridizes with the sequence set forth in SEQ ID NO:13 and a second androgen receptor GGN primer that selectively hybridizes with the sequence set forth in SEQ ID NO:14.

Also disclosed are methods wherein the first androgen receptor GGN primer has the sequence set forth in SEQ ID NO:11 and the second androgen receptor GGN primer has the sequence set forth in SEQ ID NO:12.

Further disclosed are methods wherein the AIB1 PCR product is produced using a first AIB1 primer that selectively hybridizes with the sequence set forth in SEQ ID NO:3 and a second AIB1 primer that selectively hybridizes with the sequence set forth in SEQ ID NO:4. and wherein the androgen receptor PCR product is produced using a first androgen receptor GGN primer that selectively hybridizes with the sequence set forth in SEQ ID NO:13 and a second androgen receptor GGN primer that selectively hybridizes with the sequence set forth in SEQ ID NO:14.

Also disclosed are methods wherein the first AIB1 primer has the sequence set forth in SEQ ID NO:1 and the second AIB1 primer has the sequence set forth in SEQ ID NO:2 and wherein the first androgen receptor GGN primer has the sequence set forth in SEQ ID NO:11 and the second androgen receptor GGN primer has the sequence set forth in SEQ ID NO:12.

In some embodiments the methods are performed wherein more than 29 contiguous CAG or CAA repeats in at least one allele of the AIB1 gene of the person and more than 23 contiguous GGN repeats in the androgen receptor gene of the person indicates an increased risk of prostate cancer in the subject.

In other embodiments the methods are performed wherein more than 29 contiguous CAG or CAA repeats in at least one allele of the AIB1 gene of the person and less than 23 contiguous GGN repeats in the androgen receptor gene of the person indicates an increased risk of prostate cancer in the subject.

In some embodiments the methods are performed wherein less than 29 contiguous CAG or CAA repeats in at least one allele of the AIB1 gene of the person and more than 23 contiguous GGN repeats in the androgen receptor gene of the person indicates an increased risk of prostate cancer in the subject.

In other embodiments the methods are performed wherein less than 29 contiguous CAG or CAA repeats in at least one allele of the AIB1 gene of the person and less than 23 contiguous GGN repeats in the androgen receptor gene of the person indicates an increased risk of prostate cancer in the subject.

In some embodiments the methods are performed wherein more or less than 29 contiguous CAG or CAA repeats in both alleles of the AIB1 gene of the person and more than 23 contiguous GGN repeats in the androgen receptor gene of the person indicates an increased risk of prostate cancer in the subject.

In other embodiments the methods are performed wherein more or less than 29 contiguous CAG or CAA repeats in both alleles of the AIB1 gene of the person and less than 23 contiguous GGN repeats in the androgen receptor gene of the person indicates an increased risk of prostate cancer in the subject.

In other embodiments the methods are performed wherein more than 29 contiguous CAG or CAA repeats in both alleles of the AIB1 gene of the person and more than 23 contiguous GGN repeats in the androgen receptor gene of the person indicates an increased risk of prostate cancer in the subject.

In other embodiments the methods are performed wherein less than 29 contiguous CAG or CAA repeats in both alleles of the AIB1 gene of the person and less than 23 contiguous GGN repeats in the androgen receptor gene of the person indicates an increased risk of prostate cancer in the subject.

Also disclosed are methods for assessing the risk of prostate cancer in a human subject comprising determining the length of the contiguous CAG or CAA repeats in an AIB1 gene of the subject and assessing whether the length of the CAG repeats is less than, equal to, or greater than 29 repeats, a length less than or greater than 29 repeats indicating an increased risk of prostate cancer in the subject.

Methods for assessing the risk of prostate cancer in a human subject comprising determining the length of the contiguous CAG or CAA repeats in an AIB1 gene of the subject and assessing whether the length of the CAG or CAA repeats is less than, equal to, or greater than 29 repeats, and determining the length of the contiguous CAG or CAA repeats in the androgen receptor gene of the subject and assessing whether the length of the CAG or CAA repeats is less than, equal to, or greater than 23 repeats, a length less than or greater than 29 repeats in the AIB1 allele and less than or greater than 23 repeats in the androgen receptor gene indicating an increased risk of prostate cancer in the subject are also disclosed.

Disclosed are methods for assessing the risk of prostate cancer in a human subject comprising determining the length of the contiguous CAG or CAA repeats in an AIB1 gene of the subject and assessing whether the length of the CAG or CAA repeats is less than, equal to, or greater than 29 repeats, and determining the length of the contiguous GGN repeats in the androgen receptor gene of the subject and assessing whether the length of the GGN repeats is less than, equal to, or greater than 23 repeats, a length less than or greater than 29 repeats in the AIB1 allele and less than or greater than 23 repeats in the androgen receptor gene indicating an increased risk of prostate cancer in the subject, wherein N is either T, G, or C.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

Because AR coactivators enhance transactivation of AR, the relationship of a CAG/CAA repeat length polymorphism in the AIB1/SRC-3 gene (amplified in breast cancer gene 1, a steroid receptor coactivator and an AR coactivator) with prostate cancer risk in a multidisplinary population-based case-control study in China was evaluated. Genomic DNA of 189 prostate cancer patients and 301 healthy controls was used for the PCR-based assay. The AIB1/SRC-3 CAG/CAA repeat length for all alleles ranged from 24 to 32, with the most common repeat length being 29. Homozygous 29/29 and heterozygous 28/29 were the most common genotypes, with 44% and 30% of the controls harboring these genotypes, respectively. Relative to subjects homozygous for ≧29 CAG repeats (≧29/≧29 genotype), individuals carrying one shorter allele (≧29/<29 genotype) had a 26% increased risk (OR=1.26, 95% CI=0.85-1.86), while those homozygous for the shorter allele (<29/<29 genotype) had a 73% excess risk (OR=1.73, 95% CI=0.96-3.11). The combined effect of CAG repeat lengths in the AR and AIB1/SRC-3 genes was also evaluated. Relative to men with both the ≧29/≧29 genotype of the AIB1/SRC-3 gene and a long CAG repeat length (≧23) in the AR gene, those with both the <29/<29 AIB1/SRC-3 genotype and a short CAG repeat length in the AR gene (<23) had a 2.9-fold risk (OR=2.86, 95% CI=1.28-6.40). A similar was seen for the combined effects of the AIB1/SRC-3 marker with the GGN marker of the AR gene. Together, these data indicate that the CAG/CAA repeat length in the AIB1/SRC-3 gene is associated with prostate cancer risk in Chinese men and that the combination of CAG/CAA repeat lengths in both the AIB1/SRC-3 and AR genes provide a useful marker for clinically significant prostate cancer.

### a) Materials and Methods

### (1) Study Subjects

Details of the study have been described previously (9,16-18 AIB1) which are herein are incorporated by reference for at least material related to the epidemiological study. Cases of primary prostate cancer (ICD9 185) newly diagnosed between 1993 and 1995 were identified through a rapid reporting system established between the Shanghai Cancer Institute and 16 collaborating hospitals in urban Shanghai. Cases were permanent residents in 10 urban districts of Shanghai (henceforth referred to as Shanghai) who had no history of other cancer. Contrary to many Western countries, prostate cancer screening is not widespread in China; therefore cases in this study were clinically significant prostate cancers who presented with symptoms.

Based on the personal registry cards of all adults over age 18 residing in urban Shanghai (maintained at the Shanghai Resident Registry), male population controls were selected randomly from the 6.5 million permanent residents of Shanghai and frequency-matched to the expected age distribution (5-year category) of prostate cancer cases. Included controls were negative for prostate cancer based on digital rectal exam and trans-rectal ultrasound.

Information on potential risk factors was elicited through an in-person interview by trained interviewers using a structured questionnaire. The interview included information on demographic characteristics; dietary and smoking history; consumption of alcohol and other beverages; medical history; family history of cancer; physical activity; body size; and sexual behavior. Of the 268 eligible cases (95% of the cases diagnosed in Shanghai during the study period), 243 (91%) were interviewed. After a consensus review by both the Chinese and American pathologists, four cases were classified as having benign prostatic hyperplasia and excluded from the study. Of the 495 eligible controls, 472 (95%) were interviewed. Most non-responses were due to refusal.

### b) Blood Collection and DNA Extraction

Two hundred cases (82% of those interviewed) and 330 controls (70%) provided 20 ml of fasting blood for the study. The blood samples were processed at a central laboratory in Shanghai. The buffy coat samples were first stored at −70° C. and then shipped to the U.S. in dry ice for DNA extraction at the American Type Culture Collection (Manassas, Va.), using a standard DNA extraction protocol. Quality control procedures showed no evidence of contamination, and DNA purity and length were satisfactory. After DNA extraction, 190 cases and 305 controls had sufficient DNA for genotyping. DNA samples were arranged in case-control pairs/triplets to minimize day-to-day laboratory variation, and laboratory personnel were masked to case-control status.

### c) Genotyping

The AIB1/SRC-3 gene. The polyglutamine region of the AIB1/SRC-3 protein is encoded by two glutamine codons in the AIB1/SRC-3 gene on chromosome 20 (GenBank accession number AF012108): CAG and CAA. The usual sense codon sequence of the polyglutamine stretch is (CAG)ₙ CAA (CAG)ₙ (CAA CAG)₄ CAG CAA (CAG)₂ CAA) SEQ ID NO:5. The two variable-length tracks of CAG repeats ((CAG)ₙ) usually contain six repeats between nucleotides 3930 and 3947, and nine repeats between nucleotides 3951 and 3977, for a total repeat length of 29 (19 AIB1) (which is herein incorporated by reference for material related to the CAG repeat.) This polymorphism has previously been described by Shirazi et al., though while Shirazi et al. scored genotypes of this marker using only the two variable (CAG)ₙ stretches (19 AIB1), herein the data was scored as the total number of continuous CAG and CAA triplets in the entire polyglutamine region of the AIB1/SRC-3 gene, as has been done more recently (20,21 AIB1) (both of which are herein incorporated by reference for material related to the CAG and CAA repeats).

The number of CAG/CAA repeats in the polyglutamine stretch of the AIB1/SRC-3 gene was determined by amplifying the gene's C-terminal polyglutamine region in each sample using custom flanking primers (5′-TCATCACCTCCGACAACAGAGG-3′ and 5′ (SEQ ID NO:1)-TATGGAAACTGTTGCGGAGGAG-3′ (SEQ ID NO:2) a nd the Advantage 2 Polymerase System (Clontech). The number of CAG/CAA repeats was determined by electrophoresis of the PCR products on an acrylamide gel and comparison to molecular weight standards. For confirmation, PCR products from selected samples were subsequently purified, using the PCR Product Purification Kit (Qiagen), and sequenced directly, using the Big Dye Terminator Cycle Sequencing Ready Reaction Kit (PE Biosystems).

The AR gene. Genotypes of both the CAG (polyglutamine) and GGN (polyglycine) repeat length polymorphisms in exon 1 of the AR gene (located on the X chromosome) were determined as described previously (22 AIB1) (which is herein incorporated by reference for material related to the AR gene). Briefly, two sets of oligonucleotide primers flanking each of the two polymorphic regions for use in DNA amplification and direct sequencing were designed. For the polyglutamine stretch, the number of continuous CAG or CAA triplets was counted directly, while for the polyglycine stretch, the number of continuous GGN repeats (where N represents T, G, or C) was counted directly

Quality control. Because the PCR procedure is prone to contamination, a negative water-blank control was always included in each batch of the PCR reactions (usually 9-18 DNA samples plus one negative control). If the negative control was shown to be positive, the assay was repeated for the entire batch. Twenty-four split samples from a single individual were spaced at intervals among the study samples to assess the reproducibility of genotyping. For the AIB1/SRC-3 gene, all of the 24 split samples had a CAG/CAA repeat length of 29 in both alleles. Of the 21 split samples with AR CAG results, 19 (90%) had the same repeat number (repeat length=23); one had one more, and one had one less repeat. Of the 20 samples with AR GGN results, 19 (95%) had the same repeat number (repeat length=23) and one had one less repeat.

Statistical Analysis, Unconditional logistic regression models were used to derive odds ratios (ORs) and corresponding 95% confidence intervals (CIs) to estimate the prostate cancer risks associated with AIB1/SRC-3 genotypes (23 AIB1) (which is herein incorporated by reference for material related to statistical analysis). The distribution of the number of the CAG/CAA repeat lengths among controls were used to derive the median cutoffs used to calculate the ORs. Because the AIB1/SRC-3 gene is located on chromosome 20, each individual carries two alleles. In contrast, since the AR gene is located on the X chromosome, there is only one allele for each individual. AR CAG and the AR GGN polymorphisms in this Chinese population (9 AIB1), subjects in the current AIB1/SRC-3 analysis were grouped by <23 versus ≧23 repeats in analyses stratified by each of the two AR gene polymorphisms. The level of significance for all results reported herein is 0.05.

### d) Results

Age at diagnosis ranged from 50 to 94 (median 73) for cancer cases. Due to the lack of widespread prostate cancer screening in China, cases in this study were mostly men with clinically significant prostate cancer. Accordingly, about two-thirds of the cases were diagnosed as having advanced (regional/remote stages) cancer, and most tumors were moderately or poorly differentiated. Most cases were symptomatic at diagnosis, and 77% had serum prostatic specific antigen levels greater than 10 ng/ml (median 87 ng/ml). Compared to population controls, cases had significantly higher caloric intake; had significantly larger waist-to-hip ratios; and were somewhat less likely to be married, have attended college, or be smokers or drinkers, though not significantly so (data not shown).

The distribution of the alleles and genotypes of the AIB1/SRC-3 gene CAG/CAA repeat length marker by case-control status is shown in Table 1. Among controls, the CAG/CAA repeat length for all alleles ranged from 24 to 32, with 29, 28, and 26 being the most common repeat lengths (65.6, 23.5, and 6.5%, respectively). Eighty-eight percent of the controls had at least one 29 allele. Homozygous 29/29 and heterozygous 28/29 were the most common genotypes, with 44% and 30% of the controls harboring these genotypes, respectively. The observed genotype frequencies were in close agreement with those predicted from the allele frequencies under Hardy-Weinberg equilibrium.

TABLE 1
Frequencies of AIB1/SRC-3 CAG/CAA repeat length alleles and
genotypes in prostate cancer cases and controls, China

| | |
|---|---|
| Cases | Controls |

| | n | % | n | % |
|---|---|---|---|---|
| AIB1/SRC-3 allele | | | | |
| 24 | 0 | 0.0 | 1 | 0.2 |
| 26 | 22 | 5.8 | 39 | 6.5 |
| 27 | 6 | 1.6 | 7 | 1.2 |
| 28 | 114 | 30.0 | 140 | 23.5 |
| 29 | 224 | 58.9 | 391 | 65.6 |
| 30 | 11 | 2.9 | 13 | 2.2 |
| 31 | 1 | 0.3 | 5 | 0.8 |
| 32 | 2 | 0.5 | 0 | 0.0 |
| Total | 380 | | 596 | |
| AIB1/SRC-3 genotype | | | | |
| 24/29 | 0 | 0.0 | 1 | 0.3 |
| 26/26 | 0 | 0.0 | 1 | 0.3 |
| 26/27 | 0 | 0.0 | 1 | 0.3 |
| 26/28 | 5 | 2.6 | 6 | 2.0 |
| 26/29 | 17 | 8.9 | 26 | 8.7 |
| 26/30 | 0 | 0.0 | 2 | 0.7 |
| 26/31 | 0 | 0.0 | 2 | 0.7 |
| 27/28 | 0 | 0.0 | 1 | 0.3 |
| 27/29 | 5 | 2.6 | 5 | 1.7 |
| 27/30 | 1 | 0.5 | 0 | 0.0 |
| 28/28 | 23 | 12.1 | 21 | 7.0 |
| 28/29 | 59 | 31.1 | 88 | 29.5 |
| 28/30 | 3 | 1.6 | 1 | 0.3 |
| 28/31 | 1 | 0.5 | 2 | 0.7 |
| 29/29 | 67 | 35.3 | 130 | 43.6 |
| 29/30 | 7 | 3.7 | 10 | 3.4 |
| 29/31 | 0 | 0.0 | 1 | 0.3 |
| 29/32 | 2 | 1.1 | 0 | 0.0 |
| Total | 190 | | 298 | |

Relative to men homozygous for 29 CAG/CAA repeats in the AIB1/SRC-3 gene (29/29 genotype), subjects homozygous for the 28 allele had a significant risk increase (OR=2.12, 95% CI=1.09-4.12, Table 2). Subjects with the 28/29 genotype had a non-significant increased risk relative to the 29/29 genotype (OR=1.30, 95% CI=0.83-2.03), as did men with one 29 allele and one 24, 26, or 27 allele (OR=1.33, 95% CI=0.72-2.47) and men with one 29 allele and one 30, 31, or 32 allele (OR=1.58, 95% CI=0.62-4.01).

Table 2. Age-Adjusted ORs for Prostate Cancer in Relation to CAG/CAA Repeat Lengths in the AIB1/SRC-3 Gene, China

TABLE 2
Age-adjusted ORs for prostate cancer in relation to CAG/CAA repeat
lengths in the AIB1/SRC-3 gene, China

| Allele 1 | Allele 2 | Cases | Control | OR^{a} | 95% CI |
|---|---|---|---|---|---|
| 29 | 29 | 67 | 130 | 1.00 | — |
| 28 | 29 | 59 | 88 | 1.30 | 0.83-2.03 |
| 24, 26, 27 | 29 | 22 | 32 | 1.33 | 0.72-2.47 |
| 30, 31, 32 | 29 | 9 | 11 | 1.58 | 0.62-4.01 |
| 28 | 28 | 23 | 21 | 2.12 | 1.09-4.12 |
| 26, 27, 28 | 30, 31 | 5 | 7 | 1.38 | 0.42-4.52 |
| 26, 27 | 26, 27, 28 | 5 | 9 | 1.08 | 0.35-3.35 |
| ≧29 | ≧29 | 76 | 141 | 1.00 | — |
| ≧29 | <29 | 86 | 127 | 1.26 | 0.85-1.86 |
| <29 | <29 | 28 | 30 | 1.73 | 0.96-3.11 |

Based on the median CAG/CAA repeat length of 29, the various AIB1/SRC-3 alleles were collapsed into those with 29 or more repeats (≧29) and those with less than 29 repeats (<29). Relative to men with the ≧29/≧29 genotype, men with one long and one short allele (≧89/<29 genotype) had a moderate but non-significant risk elevation (OR=1.26, 95% CI=0.85-1.86). Those with two short alleles (<29/<29 genotype) had a non-significant 73% increased risk (OR=1.73, 95% CI=0.96-3.11) relative to men with two long alleles (≧29/≧29 genotype).

Risks of prostate cancer associated with various repeat lengths in both the AIB1/SRC-3 gene CAG/CAA polymorphism as well as the two polymorphisms of the AR gene are shown in Table 3. Relative to men both homozygous for the long CAG/CAA allele (≧29/≧29 genotype) of the AIB1/SRC-3 gene and having a long AR CAG repeat length (≧23 repeats), men both homozygous for the short AIB1/SRC-3 CAG/CAA allele (<29/<29) and having a short AR CAG repeat length (<23) had a significant 2.9-fold risk (OR=2.86, 95% =1.28-6.40). Similarly, those men both homozygous for the short AIB1/SRC-3 CAG/CAA allele and having a short GGN repeat length (<23) in the AR gene had a non-significant 2.3-fold risk (OR=2.29, 95% CI=0.73-7.15) relative to men both homozygous for long AIB1/SRC-3 CAG/CAA allele (≧29/≧29 genotype) and having a long AR GGN repeat length (≧23 repeats).

TABLE 3
ORs^{a} for prostate cancer in relation to AIB/SRC-3 CAG/CAA and AR
CAG or GGN repeat lengths, China

| | CAG/CAA repeat length genotypes of the AIB1/SRC-3 gene | | | | | | | | |
|---|---|---|---|---|---|---|---|---|---|
| AR | ≧29/≧29 | | | ≧29/<29 | | | <29/<29 | | |
| polymorphisms | n1/n2^{b} | OR | 95% CI | n1/n2^{b} | OR | 95% CI | n1/n2^{b} | OR | 95% CI |
| CAG repeat | | | | | | | | | |
| length^{c} | | | | | | | | | |
| ≧23 | 30/68 | 1.00 | — | 35/69 | 1.15 | 0.63-2.08 | 9/14 | 1.46 | 0.57-3.75 |
| <23 | 45/71 | 1.43 | 0.81-2.54 | 51/58 | 1.99 | 1.12-3.53 | 19/15 | 2.86 | 1.28-6.40 |
| GGN repeat | | | | | | | | | |
| length^{c} | | | | | | | | | |
| ≧23 | 57/11 | 1.00 | — | 72/99 | 1.42 | 0.92-2.21 | 19/23 | 1.62 | 0.81-3.22 |
| | 2 | | | | | | | | |
| <23 | 17/26 | 1.28 | 0.64-2.56 | 14/24 | 1.15 | 0.55-2.40 | 7/6 | 2.29 | 0.73-7.15 |

^{a}Adjusted for age.
^{b}n1 = number of cases, n2 = number of controls.
^{c}Median number of repeats among controls was used for the cutoff.

There was no correlation between the repeat lengths in the AR and AIB1/SRC-3 genes. In addition, the number of CAG/CAA repeats in the AIB1/SRC-3 gene did not correlate with education, body mass index, waist-to-hip ratio, total caloric intake, serum levels of sex hormones (testosterone; DHT; 5α-androstane-3α,17β-diol glucuronide; and estradiol), and sex hormone binding globulin. These variables therefore were not included in the logistic model for adjustment. In addition, odds ratios were materially unchanged when the analysis was stratified by clinical stage (localized versus advanced stage disease, data not shown).

Results from this population-based case-control study in China indicate that men with fewer than 29 CAG/CAA repeats in the AIB1/SRC-3 alleles have an increased risk of clinically significant prostate cancer. Furthermore, our results suggest that this effect, though independent of AR genotypes, is more pronounced among men with a smaller number of AR CAG repeats.

The observed association with CAG/CAA repeat length in the AIB1/SRC-3 gene is biologically plausible. Data from transient transfection studies show that the AIB1/SRC-3 coactivator enhances AR transcriptional activity in the presence of DHT (12 AIB1), suggesting that the AIB1/SRC-3 coactivator, in conjunction with AR, may increase androgenic activity within the prostate gland. Amplification of the AIB1/SRC-3 gene has been implicated in the etiology of several other hormone-dependent cancers as well, including breast and ovarian cancers (24 AIB1). Furthermore, recent clinical data suggest that overexpression of AR in prostate tumors may contribute to hormone sensitivity and tumor progression (25 AIB1).

Racial/ethnic variation in the AIB1/SRC-3 CAG/CAA repeat length mirrors the risk patterns of prostate cancer in high- and low-risk populations (19,26 AIB1), thus indirectly supporting a role of AIB1/SRC-3 in prostate cancer etiology. In a small survey of 112 African Americans, 19 Chinese, and 18 Caucasians, the allele frequency of 29 CAG/CAA repeats was 61%, 76%, and 58%, respectively.

Chinese men have a longer mean CAG repeat length in the AR gene than Western men, and that a shorter AR CAG repeat length was associated with an increased risk of prostate cancer in this low-risk population (9 AIB1). The observed association with CAG/CAA repeat length in the AIB1/SRC-3 gene is independent of the AR gene: regardless of CAG repeat length in the AR gene, men homozygous for the shorter AIB1/SRC-3 allele (the <29/<29 genotype) had a higher risk than those homozygous for the long allele (≧29/≧29 genotype). However, the risk associated with the homozygous <29/<29 AIB1/SRC-3 genotype was more pronounced among those with the short AR CAG repeat length.

### 2. Example 2

### Relationship of CAG Length in the Androgen Receptor

The length of the polymorphic CAG trinucleotide repeat in the polyglutamine region of the androgen receptor (AR) gene is inversely correlated with the transactivation function of the AR. A population-based case-control assay in China addressed CAG and other polymorphisms of the AR gene and their association with clinically significant prostate cancer in this low-risk population. Genomic DNA from 190 prostate cancer patients and 304 healthy controls were used for direct sequencing to evaluate the relationship of CAG and GGN (polyglycine) repeat length in the AR gene. Relative to western men, the subjects had a longer CAG repeat length, with a median of 23 and only 10% of the subjects having a CAG repeat length shorter than 20. Men with a CAG repeat length shorter than 23 (median length) had a 65% increased risk of prostate cancer (OR=1.65, 95% CI 1.14-2.39), compared to men with a CAG repeat length of 23 or longer.

For the GGN tract (GGT₃GGG₁GGT₂GGCₙ), based on the sequencing results from 481 samples, it is shown that even though GGC regions in the polyglycine tract are highly variable, there are no mutations or polymorphisms in the GGT and GGG regions. Seventy two percent of the subjects had a GGN repeat length of 23, and those with a GGN repeat length shorter than 23 had a 12% increased risk of prostate cancer (95% CI 0.71-1.78), compared to those with 23 or more GGN repeats. This not only confirms that Chinese men do have a longer CAG repeat length than western men, but also represents the first population assay to show that even in a very low-risk population, a shorter CAG repeat length confers a higher risk of clinically significant prostate cancer. These results indicate that CAG repeat length can serve as a useful marker to identify a subset of individuals at higher risk of developing clinically significant prostate cancer.

### a) Materials and Methods

Subjects. Details of the assay have been described previously (28AR which is herein incorporate by reference for material related to the assay). Briefly, cases of primary prostate cancer (ICD9 185) newly diagnosed between 1993 and 1995 were identified through a rapid reporting system established between the Shanghai Cancer Institute (SCI) and 28 collaborating hospitals in urban Shanghai. Cases were permanent residents in 10 urban districts of Shanghai (henceforth referred to as Shanghai) who had no history of any other cancer. Of the 268 eligible cases (representing 95% of the cases diagnosed in urban Shanghai during the study period), 243 (91%) were interviewed in person by trained interviewers. Four of the cases were later classified as having benign prostatic hyperplasia and excluded from the study after a consensus review by both Chinese and U.S. pathologists.

Based on the records at the Shanghai Resident Registry, which contains personal identification cards for all adult residents over age 18 in urban Shanghai, healthy subjects who were free of cancer were selected randomly from permanent residents of Shanghai (6.5 million), frequency-matched to the expected age distribution (5-year category) of prostate cancer cases. Of the 495 eligible controls without a history of cancer, 472 (95%) were interviewed.

Information on potential risk factors was elicited through an in-person interview by trained interviewers using a structured questionnaire. The interview included information on demographic characteristics; dietary history; smoking history; consumption of alcohol and other beverages; medical history; family history of cancer; physical activity; body size; and sexual behavior.

Blood collection and DNA extraction. Two hundred cases (84% of those interviewed) and 330 controls (70%) provided 20 ml of fasting blood for the study. The blood samples were processed within three hours of collection at a central laboratory in Shanghai and stored at −70° C. The frozen buffy coat samples (separated from 5 ml of blood) were later shipped to the U.S. on dry ice for DNA extraction at the American Type Culture Collection (Manassas, Va.) with standard protocols. DNA purity, yield, and length were satisfactory and there was no evidence of DNA degradation or RNA contamination. Following DNA extraction, 191 cases and 304 controls had sufficient DNA for AR genotyping at the University of Rochester. DNA samples for cases and controls were grouped into pairs to minimize the effect of day-to-day laboratory variation. Laboratory personnel were blinded to the case-control status.

Molecular analysis and assessment of the CAG and GGN repeats. As part of an ongoing molecular analysis of the AR gene, genomic DNA from the 495 subjects was used to determine the usual sense codon sequence and the exact number of CAG and GGN repeats in exon 1 of the AR gene through PCR amplification and DNA sequencing. For the CAG repeat analysis, a set of oligonucleotide primers that flank the CAG repeat, 5′-GCTCTGGGACGCAA-CCTCTCT-3′(SEQ ID NO:7) and 5′-GCAGCGACTACCGCATCATCA-3′(SEQ ID NO:8), were designed for PCR amplification. A pair of nested primers, 5′-CGGG-GTAAGGGAAGTAGGTGGAAG-3′(SEQ ID NO:15), and 5′-CTCTACGATGGGCTTGGGGAGAAC-3′ (SEQ ID NO:16) was selected for DNA sequencing. For GGN analysis, the oligonucleotide primers 5′-ACCCTCAGCCGCCGCTTCCTCATC-3′ (SEQ ID NO:11)and 5′-CTGGGATAGGGCACTCTGCTCAAC-3′ (SEQ ID NO:12) were used for both PCR amplification and sequencing. The PCR products of the CAG and GGN repeats were amplified, using the Advantage 2 Polymerase System (Clontech) and the Advantage-GC Genomic Polymerase System (Clontech), respectively. Subsequently, these PCR products were purified, using the PCR Product Purification Kit (Qiagen), and sequenced directly, using the Big Dye Terminator Cycle Sequencing Ready Reaction Kit (PE Biosystems). All reactions were optimized to reach consistent results, using genomic DNA samples extracted from cell lines. For the polyglutamine tract ((CAG)ₙCAA), the number of CAG triplets was counted to yield the length of CAG repeats. For the polyglycine tract (GGT₃GGG₁GGT₂GGCₙ) (SEQ ID NO:17), the usual sense codon sequence of the GGN tract is: three GGT, one GGG, two GGT, followed by a variable number of GGC repeats. For example, a GGN repeat length of 23 in our study corresponded to a PCR fragment of 217 bp, encompassing 3 GGT, 1 GGG, 2 GGT, and 17 GGC triplets.

Because the PCR procedure is prone to contamination, a negative control (water blank) was always included in each batch of PCR reactions (usually 9-18 samples plus one negative control). The assay for one batch (9 samples) was repeated with new reagents because of an indication of minor contamination. Because exon 1 of the AR gene is GC-rich with CAG and GGN repeats, this region is difficult to amplify. Several samples had to be amplified and sequenced more than once. Overall, five (1%) of the 495 samples could not be typed for CAG repeats due to insufficient DNA or sequencing problems, while 14 (2.8%) could not be typed for GGN repeats for similar reasons. The percentages of samples that were unsuccessfully genotyped were similar in cases and controls.

Twenty-four split samples from the same individual were included as quality control samples to assess the reproducibility of genotyping. Of the 24 quality control samples, 23 and 20 were amplified and sequenced successfully for the CAG and GGN repeats, respectively. Of the 23 samples with CAG results, 21 (91%) had the same repeat length of 23, one had 24, and one had 22. Of the 20 samples with GGN results, 19 (95%) had the same repeat length of 23 and the other had length 24.

Statistical Analysis. The mean numbers of CAG and GGN repeats were compared in cases and controls using the t test. Unconditional logistic regression models were used to estimate odds ratios (ORs) and their corresponding 95 percent confidence intervals (CIs) for prostate cancer in relation to CAG and GGN repeat lengths (29AR). Repeat lengths were examined first as continuous variables and later as categorical variables. The distributions of the number of CAG or GGN repeats among controls were used to derive the median or tertile cutoffs used to calculate ORs. In addition, the combined effects of CAG and GGN were evaluated based on the median lengths within the controls. The relationships between age, CAG and GGN repeat length, and other variables were assessed by Spearman correlation and analysis of variance.

### b) Results

Selected characteristics of cases and controls are shown in Table 4. Compared to controls, cases had higher caloric intake and higher levels of education and waist-to-hip ratio and were less likely to use cigarettes or alcohol. Age at diagnosis ranged from 50 to 94 (median 73) for cancer cases. Sixty-nine cases (36%) were diagnosed as having localized cancer, and most tumors (72%) were moderately or poorly differentiated.

TABLE 4
Selected characteristics of prostate cancer cases
and population controls, China

| | Cases (n = 191) | | Controls (n = 304) | |
|---|---|---|---|---|
| Characteristics | Mean | (S.D.) | Mean | (S.D.) |
| Age (yrs) | 72.2 | (7.7) | 71.9 | (7.3) |
| Total calories (Kcal/day) | 2457.0 | (647) | 2342.0 | (731) |
| Height (cm) | 167.9 | (6.0) | 167.6 | (5.8) |
| Weight (Kg) | 61.3 | (8.4) | 61.5 | (10.1) |
| BMI (Kg/m2) | 21.8 | (2.9) | 21.9 | (3.3) |
| Waist circumference (cm) | 82.6 | (10.4) | 82.5 | (10.7) |
| Hip circumference (cm) | 90.7 | (8.9) | 92.6 | (8.5) |
| Waist-to-hip ratio | 0.91 | (0.05) | 0.89 | (0.05) |
| % married | 89.5 | | 92.1 | |
| % with education greater | 34.6 | | 25.3 | |
| than high school | | | | |
| % smokers | 56.5 | | 65.1 | |
| % alcohol users | 31.4 | | 42.1 | |
| Clinical stage (%) | | | | |
| Localized | 36.3 | | | |
| Regional | 30.5 | | | |
| Remote | 32.1 | | | |
| Histologic grade (%) | | | | |
| Well differentiated | 7.9 | | | |
| Moderately differentiated | 31.0 | | | |
| Poorly differentiated | 41.0 | | | |
| Cannot be assessed | 20.1 | | | |

Because the AR gene is located on the X chromosome, only one copy of the gene is present in men. For the polyglutamine tract (CAGₙCAA), there was no variation in the CAA sequence among the 490 samples analyzed. The number of CAG repeats ranged from 10 to 34. About 65% of the study subjects had a CAG repeat length that ranged from 21 to 24, but only 1% of the subjects had a CAG length longer than 30 repeats (Table 5).

TABLE 5
Distribution of number of CAG repeats in the androgen
receptor gene in prostate cancer cases and controls, China

| | |
|---|---|
| Cases | Controls |
| (n = 190) | (n = 300) |

| No. of CAG repeats | N | % | N | % |
|---|---|---|---|---|
| 10 | 0 | 0.0 | 1 | 0.3 |
| 14 | 0 | 0.0 | 1 | 0.3 |
| 15 | 4 | 2.1 | 2 | 0.7 |
| 16 | 3 | 1.6 | 2 | 0.7 |
| 17 | 1 | 0.5 | 3 | 1.0 |
| 18 | 7 | 3.7 | 9 | 3.0 |
| 19 | 7 | 3.7 | 12 | 4.0 |
| 20 | 13 | 6.8 | 17 | 5.7 |
| 21 | 24 | 12.6 | 32 | 10.7 |
| 22 | 57 | 30.0 | 67 | 22.3 |
| 23 | 22 | 11.6 | 46 | 15.3 |
| 24 | 21 | 11.1 | 48 | 16.0 |
| 25 | 16 | 8.4 | 19 | 6.3 |
| 26 | 9 | 4.7 | 17 | 5.7 |
| 27 | 3 | 1.6 | 15 | 5.0 |
| 28 | 2 | 1.1 | 2 | 0.7 |
| 29 | 0 | 0.0 | 3 | 1.0 |
| 30 | 0 | 0.0 | 1 | 0.3 |
| 31 | 0 | 0.0 | 2 | 0.7 |
| 32 | 0 | 0.0 | 0 | 0.0 |
| 33 | 0 | 0.0 | 1 | 0.3 |
| 34 | 1 | 0.5 | 0 | 0.0 |
| Median | 22 | | 23 | |

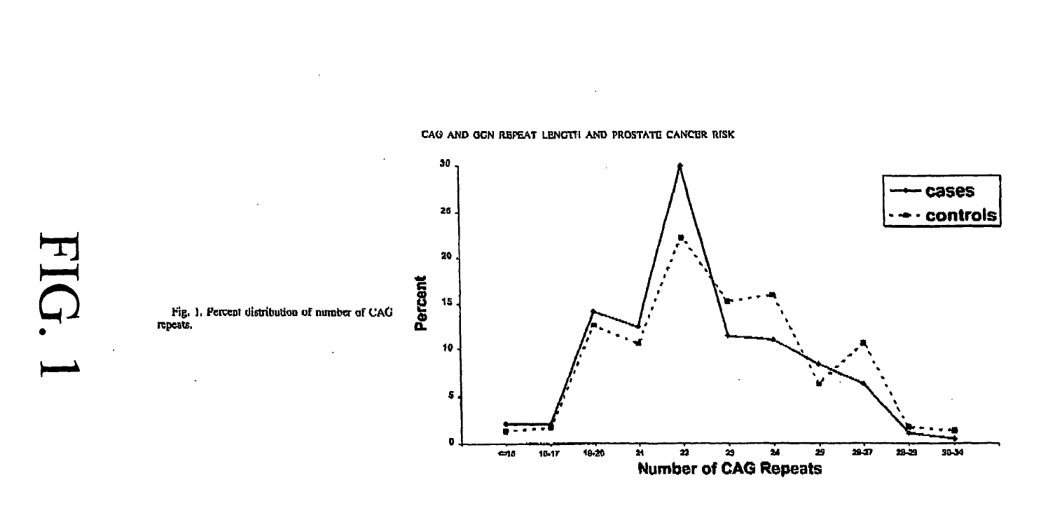

Although the median number of CAG repeats in controls was only slightly larger than that in cases (23.0 vs. 22.0), there was a shift toward longer repeat length among controls (FIG. 1). For CAG repeat length shorter than 23, cases had higher percentages than controls in 6 of the 10 categories. However, for CAG repeat length longer than 22, controls had higher percentage than cases in 8 of the 12 categories. Age at diagnosis and stage of cancer were not related to CAG repeat length, with similar distribution and average number of CAG and GGN repeat lengths in various age categories and three clinical stages.

For the polyglycine tract (GGT₃GGG₁GGT₂GGCₙ) (SEQ ID NO:17), there was no variation in the codon usage or the number of GGT and GGG trinucleotides in all of the 481 samples analyzed, although the number of GGC repeats was highly variable. The pattern was always three GGT, one GGG, two GGT, followed by a variable number of GGC. The number of GGN repeats among study subjects ranged from 15 to 27 (the number of GGC repeats thus ranged from 9 to 21) (Table 6). About 72% of the study subjects had a GGN repeat length of 23.

TABLE 6
Distribution of number of GGN repeats in the androgen
receptor gene in prostate cancer cases and controls, China

| | |
|---|---|
| Cases | Controls |
| (n = 187) | (n = 295) |

| No. of GGN repeats | N | % | N | % |
|---|---|---|---|---|
| 14 | 0 | 0.0 | 1 | 0.3 |
| 15 | 1 | 0.5 | 1 | 0.3 |
| 16 | 2 | 1.1 | 1 | 0.3 |
| 17 | 2 | 1.1 | 0 | 0.0 |
| 18 | 0 | 0.0 | 1 | 0.3 |
| 19 | 19 | 10.2 | 20 | 6.8 |
| 20 | 2 | 1.1 | 2 | 0.7 |
| 21 | 3 | 1.6 | 0 | 0.0 |
| 22 | 10 | 5.3 | 24 | 8.2 |
| 23 | 136 | 72.7 | 212 | 72.1 |
| 24 | 10 | 5.3 | 24 | 8.2 |
| 25 | 2 | 1.1 | 1 | 0.3 |
| 27 | 0 | 0.0 | 1 | 0.3 |
| Median | 23 | | 23 | |

Risks of prostate cancer associated with CAG and GGN repeat lengths are shown in Table 7. When the number of CAG repeats was included in the model as a continuous variable, there was a 7% increase in the risk of prostate cancer for each decrement in length of one CAG repeat (OR=1.07, 95% CI 1.00-1.15). The risks associated with decrements of three and six repeats were 1.21 (95% 1.14-1.32) and 1.42 (95% CI 1.22-1.61), respectively. When the median repeat length was used to dichotomize study subjects, men with a CAG repeat length shorter than 23 had a 65% increased risk (OR=1.65, 95% CI 1.14-2.39), compared to men with a CAG repeat length of 23 or longer. Relative to the highest tertile of CAG repeat length (≧24), men in the second and first tertiles (22-23 and <22) had ORs of 1.45 and 1.55, respectively (P_{trend}=0.06).

TABLE 7
Odds ratios (ORs)^{a} and 95% confidence intervals (CIs) for prostate
cancer in relation to the number of GAG and GGN repeats in the
androgen receptor gene, China

| Number of CAG | Cases | Controls | | |
|---|---|---|---|---|
| and GGN repeats | No. | No. | ORa | 95% CI |
| No. of CAG repeats | | | | |
| Continuous (per decrement | 190 | 300 | 1.07 | 1.00-1.15 |
| of one CAG repeat) | | | | |
| Median | | | | |
| ≧23 | 74 | 154 | 1.00 | — |
| <23 | 116 | 146 | 1.65 | 1.14-2.39 |
| Tertile | | | | |
| ≧24 | 52 | 108 | 1.00 | — |
| 22-23 | 79 | 113 | 1.45 | 0.93-2.25 |
| <22 | 59 | 79 | 1.55 | 0.96-2.49 |
| | | | Linear trend | p = 0.06 |
| No. of GGN repeats | | | | |
| Continuous (per decrement | 187 | 294 | 1.07 | 0.96-1.20 |
| of one GGN repeat) | | | | |
| Median | | | | |
| ≧23 | 147 | 239 | 1.00 | 1.00 |
| <23 | 39 | 56 | 1.12 | 0.71-1.78 |
| Combined number of CAG | | | | |
| and GGN repeats | | | | |
| CAG ≧ 23, GGN ≧ 23 | 53 | 120 | 1.00 | — |
| CAG ≧ 23, GGN < 23 | 19 | 29 | 1.48 | 0.76-2.88 |
| CAG < 23, GGN > 23 | 94 | 115 | 1.85 | 1.21-2.82 |
| CAG < 23, GGN < 23 | 20 | 26 | 1.75 | 0.90-3.41 |

^{a}Adjusted for age (continuous).

Similarly, men with a shorter GGN repeat length had a higher risk of prostate cancer. Each decrement of one GGN repeat length was associated with a 7% increase in risk (OR=1.07, 95% CI 0.96-1.20). Men with a GGN repeat length shorter than the median length of 23 had a 12% increase in prostate cancer risk, compared to those with 23 or more repeats. Because more than 72% of the subjects had 23 GGN repeats, the ORs by tertiles for GGN repeats were not estimated.

Also shown in Table 7 are the ORs associated with combined categories of CAG and GGN repeat lengths. Men with both CAG and GGN repeat lengths shorter than 23 had a 75% elevated risk of prostate cancer. There was little correlation between the number of CAG and GGN repeats (r=−0.03, p>0.05).

The number of CAG or GGN repeats did not correlate with age, education, body mass index, waist-to-hip ratio, total calories, smoking, or drinking. These variables therefore were not included in the model for adjustment. The ORs were materially unchanged after further adjustment for benign prostatic hyperplasia (BPH), although the cases had a higher prevalence of BPH (57% vs. 23%) and there was a non-significant moderate association between CAG or GGN repeat lengths and BPH (data are reported separately). Associations of CAG or GGN repeat length were similar across all stages of disease at diagnosis (data not shown).

These results confirm that a shorter CAG repeat length is associated with an increased risk of clinically significant prostate cancer. A shorter length of GGN repeat also appears to increase the risk of prostate cancer, but the magnitude of excess risk was smaller.

The observed inverse association with AR polymorphisms is biologically plausible, as laboratory studies have shown that a long polyglutamine chain (>30 repeats) in the AR gene is associated with androgen insensitivity and reduced AR transactivation activity (13,14AR). In vitro transfection studies also have demonstrated that elimination of the polyglutamine tracts results in elevated transcriptional activities (9,11,16AR). Clinical studies have suggested that alteration in the AR function, either through polymorphisms of CAG repeat length or somatic mutations, may be associated with tumor progression. For example, the progression from latent to clinically invasive prostate cancer is initially androgen-dependent, although some tumors later become androgen-independent (thus becoming non-responsive to hormonal treatment). Several non-germline-related changes of the AR gene, including amplification of the AR gene (usually a key step in the transition from a hormone-sensitive to a hormone-refractory state in prostate tumors) (31,32AR), AR somatic mutations (identified throughout transactivation, DNA binding, and ligand binding domains) (33,34AR), and contraction of CAG repeat length in cancer cells (32AR), have been shown to be associated with tumor aggressiveness, cancer progression, and failure of hormonal therapy. AR expression studies in the majority of prostate tumors, including those that have become refractory to hormonal therapy, also suggest that AR plays a key role in androgen-independent tumors (35,36AR).

The inverse relationship between CAG repeat length and AR transcriptional activity (thus androgen sensitivity) is the currently recognized underlying molecular mechanism by which AR polymorphisms modulate prostate cancer risk. Because transcriptional activation of the AR gene is influenced by not only polymorphisms in the AR gene but also a number of other factors, including tissue levels of dihydrotestosterone (DHT), estradiol, insulin-like growth factors, and AR coactivators (37-43AR), it is likely that these factors may also affect prostate cancer risk by mediating transcriptional activities. Several AR coactivators, including AR-associated proteins (ARA70, ARA55), AIB1 (Amplified in Breast Cancer-1), CBP (cyclic AMP responsive element binding protein), Rb, and BRCA1, have been shown to enhance AR-mediated transcriptional activity from 2- to 10-fold, suggesting that in vivo coactivators are essential in attaining optimal AR transactivation in response to androgens (40-43AR).

It has been suggested that variations in CAG repeat length in the AR gene between populations may explain part of the large racial difference in prostate cancer risk and that a shorter CAG repeat length reported for African Americans may contribute to some of their higher risk of prostate cancer, although currently no data are available from this population. Our results confirm that, relative to western men, Chinese men do indeed have a longer CAG repeat length. For example, 22% of the 1,722 white men in two U.S. studies (17,18AR) had a CAG repeat length shorter than 20 vs. only 10% in our study and 55% reported for African Americans in a cross-sectional survey (26,27AR). Inverse associations have also been reported for Caucasians, suggesting that the underlying biological mechanism in various racial groups may be similar and that the polymorphisms of AR may be related, in part, to racial difference in prostate cancer risk.

The common polymorphism of the AR gene confers variable risk upon all individuals, which in turn may result in a much larger proportion of prostate cancer cases attributable to having fewer CAG repeats. Assuming that the CAG polymorphism association is causal, it is estimated that 25% (95% CI 9% to 41%) of the cases in Shanghai can be attributed to a CAG repeat length shorter than 23. Using the CAG repeat length distribution in the two U.S. studies among white men (17,18AR), it was estimated that 3-7%% of cases in the U.S. white men can be attributed to the CAG polymorphism (repeat length <23) and that this polymorphism alone potentially accounts for at least 5% of the difference in incidence between Chinese and U.S. men.

Similar to two previous studies (17,18AR), it was found that the number of GGN repeats clusters around 23 (in the study of Stanford et al., only the number of GGC repeats was counted and 15 was the peak of the repeat, which corresponds to 21 GGN repeats), and that a shorter GGN repeat length appears to be associated with a moderate increase in prostate cancer risk. Twenty-three GGN repeats may represent the coding sequence for optimal AR protein conformation and activity, because over 70% of the study subjects in our study as well as in studies of western men had a GGN repeat length of 23.

Although it is well established that (GGC)ₙ repeats in the polyglycine tract (GGT₃GGG₁GGT₂GGCₙ) (SEQ ID NO:17) of the AR gene is polymorphic, to date there has been little information on variations in the GGG and GGT regions of the polyglycine tract, because these regions are GC-rich and technically it has been difficult to amplify these regions. Our study represents the first successful effort to sequence the exact codon usage and number of the GGN trinucleotide repeats in a large number of population-based samples. It was showed that GGT and GGG regions were quite stable and there were no variations in these two regions in all of the 481 DNA samples analyzed.

### F. AIB1 REFERENCES

Reference numbers throughout the application that have an “AIB1” associated with them refer to this list of references.

1. Hsing, A. W., Devesa, S. S., Jin, F., and Gao, Y. T. Rising incidence of prostate cancer in Shanghai, China. Cancer Epidemiol Biomarkers Prev, 7: 83-84, 1998.

2. Hsing, A. W., Tsao, L., and Devesa, S. S. International trends and patterns of prostate cancer incidence and mortality. Int J Cancer, 85: 60-67, 2000.

3. Hsing, A. W. and Devesa, S. S. Trends and patterns of prostate cancer: what do they suggest? Epidemiologic Reviews, 2001. (in press)

4. Ross, R. K., Pike, M. C., Coetzee, G. A., Reichardt, J. K., Yu, M. C., Feigelson, H., Stanczyk, F. Z., Kolonel, L. N., and Henderson, B. E. Androgen metabolism and prostate cancer: establishing a model of genetic susceptibility. Cancer Res, 58: 4497-4504, 1998.

5. Ross, R. K., Bernstein, L., Lobo, R. A., Shimizu, H., Stanczyk, F. Z., Pike, M. C., and Henderson, B. E. 5-alpha-reductase activity and risk of prostate cancer among Japanese and US white and black males. Lancet, 339: 887-889, 1992.

6. Shibata, A. and Whittemore, A. S. Genetic predisposition to prostate cancer: possible explanations for ethnic differences in risk. Prostate, 32: 65-72, 1997.

7. Stanford, J. L., Just, J. J., Gibbs, M., Wicklund, K. G., Neal, C. L., Blumenstein, B. A., and Ostrander, E. A. Polymorphic repeats in the androgen receptor gene: molecular markers of prostate cancer risk. Cancer Res, 57: 1194-1198, 1997.

8. Giovannucci, E., Stampfer, M. J., Krithivas, K., Brown, M., Dahl, D., Brufsky, A., Talcott, J., Hennekens, C. H., and Kantoff, P. W. The CAG repeat within the androgen receptor gene and its relationship to prostate cancer. Proc Natl Acad Sci U S A, 94: 3320-3323, 1997.

9. Hsing, A. W., Gao, Y. T., Wu, G., Wang, X., Deng, J., Chen, Y. L., Sesterhenn, I. A., Mostofi, F. K., Benichou, J., and Chang, C. Polymorphic CAG and GGN repeat lengths in the androgen receptor gene and prostate cancer risk: a population-based case-control study in China. Cancer Res., 60: 5111-5116, 2000.

10. Yeh, S., Miyamoto, H., Shima, H., and Chang, C. From estrogen to androgen receptor: a new pathway for sex hormones in prostate. Proc Natl Acad Sci U S A, 95: 5527-5532, 1998.

11. Heinlein, C. A., Ting, H. J., Yeh, S., and Chang, C. Identification of ARA70 as a ligand-enhanced coactivator for the peroxisome proliferator-activated receptor gamma. J Biol Chem, 274: 16147-16152, 1999.

12. Yeh, S., Kang, H. Y., Miyamoto, H., Nishimura, K., Chang, H. C., Ting, H. J., Rahman, M., Lin, H. K., Fujimoto, N., Hu, Y. C., Mizokami, A., Huang, K. E., and Chang, C. Differential induction of androgen receptor transactivation by different androgen receptor coactivators in human prostate cancer DU145 cells. Endocrine., 11: 195-202, 1999.

13. Yeh, S. and Chang, C. Cloning and characterization of a specific coactivator, ARA70, for the androgen receptor in human prostate cells. Proc. Natl. Acad. Sci. U. S. A, 93: 5517-5521, 1996.

14. Anzick, S. L., Kononen, J., Walker, R. L., Azorsa, D. O., Tanner, M. M., Guan, X. Y., Sauter, G., Kallioniemi, O. P., Trent, J. M., and Meltzer, P. S. AIB1, a steroid receptor coactivator amplified in breast and ovarian cancer. Science, 277: 965-968, 1997.

15. Bautista, S., Valles, H., Walker, R. L., Anzick, S., Zeillinger, R., Meltzer, P., and Theillet, C. In breast cancer, amplification of the steroid receptor coactivator gene AIB1 is correlated with estrogen and progesterone receptor positivity. Clin Cancer Res, 4. 2925-2929, 1998.

16. Hsing, A. W., Chen, C., Chokkalingam, A. P., Gao, Y. T., Dightman, D. A., Nguyen, H. T., Deng, J., Cheng, J., Sesterhenn, I. A., Mostofi, F. K., Stanczyk, F. Z., and Reichardt, J. K. V. Polymorphic markers in the SRD5A2 gene and prostate cancer risk: a population-based case-control study. Cancer Epidemiol Biomarkers Prev, 2001. (in press)

17. Hsing, A. W., Deng, J., Sesterhenn, I. A., Mostofi, F. K., Stanczyk, F. Z., Benichou, J., Xie, T., and Gao, Y. T. Body size and prostate cancer: a population-based case-control study in China. Cancer Epidemiol. Biomarkers Prev., 9: 1335-1341, 2000.

18. Hsing, A. W., Chua, S., Jr., Gao, Y. T., Gentzschein, E., Chang, L., Deng, J., and Stanczyk, F. Z. Prostate cancer risk and serum levels of insulin and leptin: a population-based study. J. Natl. Cancer Inst., 93: 783-789, 2001.

19. Shirazi, S. K., Bober, M. A., and Coetzee, G. A. Polymorphic exonic CAG microsatellites in the gene amplified in breast cancer (AIB1 gene). Clin Genet, 54: 102-103, 1998.

20. Platz, E. A., Giovannucci, E., Brown, M., Cieluch, C., Shepard, T. F., Stampfer, M. J., and Kantoff, P. W. Amplified in breast cancer-1 glutamine repeat and prostate cancer risk. Prostate J., 2: 27-32, 2000.

21. Rebbeck, T. R., Wang, Y., Kantoff, P. W., Krithivas, K., Neuhausen, S. L., Godwin, A. K., Daly, M. B., Narod, S. A., Brunet, J. S., Vesprini, D., Garber, J. E., Lynch, H. T., Weber, B. L., and Brown, M. Modification of brca1- and brca2-associated breast cancer risk by aib1 genotype and reproductive history. Cancer Res., 61: 5420-5424, 2001.

22. Chang, C. S., Kokontis, J., and Liao, S. T. Structural analysis of complementary DNA and amino acid sequences of human and rat androgen receptors. Proc. Natl. Acad. Sci. U. S. A, 85: 7211-7215, 1988.

23. Breslow, N. E. and Day, N. E. Statistical methods in cancer research. Volume I - The analysis of case-control studies. IARC Sci Publ, 32: 5-338, 1980.

24. Thenot, S., Charpin, M., Bonnet, S., and Cavailles, V. Estrogen receptor cofactors expression in breast and endometrial human cancer cells. Mol. Cell Endocrinol., 156: 85-93, 1999.

25. Bubendorf, L., Kononen, J., Koivisto, P., Schraml, P., Moch, H., Gasser, T. C., Willi, N., Mihatsch, M. J., Sauter, G., and Kallioniemi, O. P. Survey of gene amplifications during prostate cancer progression by high-throughout fluorescence in situ hybridization on tissue microarrays. Cancer Res., 59: 803-806, 1999.

26. Hayashi, Y., Yamamoto, M., Ohmori, S., Kikumori, T., Imai, T., Funahashi, H., and Seo, H. Polymorphism of homopolymeric glutamines in coactivators for nuclear hormone receptors. Endocr J. 46: 279-284, 1999.

27. McKenna, N. J., Xu, J., Nawaz, Z., Tsai, S. Y., Tsai, M. J., and O'Malley, B. W. Nuclear receptor coactivators: multiple enzymes, multiple complexes, multiple functions. J. Steroid Biochem. Mol. Biol., 69: 3-12, 1999.

28. Heery, D. M., Kalkhoven, E., Hoare, S., and Parker, M. G. A signature motif in transcriptional co-activators mediates binding to nuclear receptors. Nature, 387: 733-736, 1997.

29. Smith, J. R., Freije, D., Carpten, J. D., Gronberg, H., Xu, J., Isaacs, S. D., Brownstein, M. J., Bova, G. S., Guo, H., Bujnovszky, P., Nusskern, D. R., Damber, J. E., Bergh, A., Emanuelsson, M., Kallioniemi, O. P., Walker-Daniels, J., Bailey-Wilson, J. E., Beaty, T. H., Meyers, D. A., Walsh, P. C., Collins, F. S., Trent, J. M., and Isaacs, W. B. Major susceptibility locus for prostate cancer on chromosome 1 suggested by a genome-wide search. Science, 274: 1371-1374, 1996.

### G. AR REFERENCES

Reference numbers throughout the application that have an “AR” associated with them refer to this list of references.

1. Parkin, D. M., Whelan, S. L., Ferlay, J., Raymond, L., and Young, L. (eds). Cancer incidence in Five Continents. Vol II. IARC Scientific Publications, 143, International Agency for Research on Cancer, Lyon, 1997.

2. Hsing, A. W., Tsao, L., and Devesa, S. S. International trends and patterns of prostate cancer incidence and mortality. Int. J. Cancer, 85: 60-67, 2000.

3. Hsing, A. W., Devesa, S. S., Jin, F., and Gao, Y.-T. Rising incidence of prostate cancer in Shanghai, China. Cancer Epidemiol. Biomarkers Prev., 7: 83-84, 1998.

4. Shimizu, H., Ross, R. K., and Bernstein, L. Possible underestimation of the incidence rate of prostate cancer in Japan. Jpn. J. Cancer Res., 82: 483-485, 1991.

5. Breslow, N., Chan, C. W., Dhom, G., Drury, R. A., Franks, L. M., Gellei, B., Lee, Y. S., Lundberg, S., Sparke, B., Stemby, N. H., and Tulinius, H. Latent carcinoma of the prostate of autopsy in seven areas. Int. J. Cancer, 20: 680-688, 1977.

6. Roy, A. K., Lavrovsky, Y., Song, C. S., Chen, S., Jung, M. H., Velu, N. K., Bi, B. Y., and Chatteiee, B. Regulation of androgen action. Vitam. Horm., 55: 309-352, 1999.

7. Trapman, J., and Brinkmann, A. O. The androgen receptor in prostate cancer. Path. Res. Pract., 192: 752-760, 1996.

8. McPhaul, M. J. Molecular defects of the androgen receptor. J. Steroid Biochem. Mol. Biol., 69: 315-322,1999.

9. Chamberlain, N. L., Driver, E. D., and Miesfeld, R. L. T. The length and location of CAG trinucleotide repeats in the androgen receptor N-terminal domain affect transactivation function. Nucleic Acids Res., 22: 3181-3186, 1994.

10. Choong, C. S., Kemppainen, J. A., Zhou, Z. X., and Wilson, E. M. Reduced androgen receptor gene expression with first exon CAG repeat expansion. Mol. Endocrinol., 10: 1527-1535, 1996.

11. Tut, T. G., Ghadessy, F. J., Trifiro, M. A., Pinsky, L., and Yong, E. L. Long polyglutamine tracts in the androgen receptor are associated with reduced trans-activation, impaired sperm production, and male infertility. J. Clin. Endocrinol. Metab., 82: 3777-3782, 1997.

12. Zirkin B. R. Spermatogenesis: its regulation by testosterone and FSH. Semin. Cell Dev. Biol., 9: 417-421, 1998.

13. Fischbeck, K. H., Lieberman, A., Bailey, C. K., Abel, A., and Merry D. E. Androgen receptor mutation in Kennedy's disease. Philos. Trans. R. R. Soc. Lond. B. Biol. Sci., 354: 1075-1078, 1999.

14. Brooks, B. P., and Fischbeck, K. H. Spinal and bulbar muscular atrophy: a trinucleotide-repeat expansion neurodegenerative disease. Trends Neurosci., 18: 459-461, 1995.

15. Kazemi-Esfarjani, P., Trifiro, M. A., and Pinsky, L. Evidence for a repressive function of the long polyglutamine tract in the human androgen receptor: possible pathogenetic relevance for the (CAG)n-expanded neuronopathies. Hum. Mol. Genet., 4: 523-527, 1995.

16. Gao, T., Marcelli, M., and McPhaul, M. J. Transcriptional activation and transient expression of the human androgen receptor. J. Steroid Biochem. Mol. Biol., 59:9-20, 1996.

17. Stanford, J. L., Just, J. J., Gibbs, M., Wicklund, K. G., Neal, C. L., Blumenstein, B. A., and Ostrander, E. A. Polymorphic repeats in the androgen receptor gene: molecular markers of prostate cancer risk. Cancer Res., 57: 1194-1198, 1997.

18. Giovannucci, E., Stampfer, M. J., Krithivas, K., Brown, M., Dahl, D., Brufsky, A., Talcott, J., Hennekens, C. H., and Kantoff, P. W. The CAG repeat within the androgen receptor gene and its relationship to prostate cancer. Proc. Natl. Acad. Sci., 94: 3320-3323, 1997.

19. Ingles, S. A., Ross, R. K., Yu, M. C., Irvine, R. A., La Pera, G., Haile, R. W., and Coetzee, G. A Association of prostate cancer risk with genetic polymorphisms in vitamin D receptor and androgen receptor. J. Natl. Cancer Inst., 89: 166-170, 1997.

20. Platz, E. A., Giovannucci, E., Dahl, D. M., Krithivas, K., Hennekens, C. H., Brown, M., Stampfer, M. J., and Kantoff, P. W. The androgen receptor gene GGN microsatellite and prostate cancer risk. Cancer Epidemiol. Biomarkers Prev., 7: 379-384, 1998.

21. Kantoff, P., Giovannucci, E., and Brown, M. The androgen receptor CAG repeat polymorphism and its relationship to prostate cancer. Biochim. Biophys. Acta., 1378: C1-C5, 1998.

22. Hakimi, J. M., Schoenberg, M. P., Rondinelli, R. H., Piantadosi, S., and Barrack, E. R. Androgen receptor variants with short glutamine or glycine repeats may identify unique subpopulations of men with prostate cancer. Clin. Cancer Res., 3: 1599-1608, 1997.

23. Sartor, O., Zheng, Q., and Eastham, J. A. Androgen receptor gene CAG repeat length varies in a race-specific fashion in men without prostate cancer. Urology, 53: 378-380, 1999.

24. Hardy, D. O., Scher, H. I., Bogenreider, T., Sabbatini, P., Zhang, Z. F., Nanus, D. M., and Catterall, J. F. Androgen receptor CAG repeat lengths in prostate cancer: correlation with age of onset. Clin. Endocrinol. Metab., 81: 4400-4405, 1996.

25. Bratt, O., Borg, A., Kristoffersson, U., Lundgren, R., Zhang, Q. X., Olsson, H. CAG repeat length in the androgen receptor gene is related to age at prostate cancer diagnosis and response to endocrine therapy, but not to risk of prostate cancer. Br. J. Cancer, 81: 672-676, 1999.

26. Irvine, R. A., Yu, M. C., Ross, R. K., and Coetzee, G. A. The CAG and GGC microsatellites of the androgen receptor gene are in linkage disequilibrium in men with prostate cancer. Cancer Res., 55: 1937-1940, 1995.

27. Edwards A, Hammond H A, Jin L, Caskey C T, Chakraborty R. Genetic variation at five trimeric and tetrameric tandem repeat loci in four human population groups. Genomics;12: 241-253, 1992.

28. Hsing, A. W., Deng, J., Tong, X., Sesterhenn, I., Mostofi, F. K., and Gao, Y-T. Body size and prostate cancer: a population-based case-control study in Shanghai, China (Submitted).

29. Breslow, N. E. and Day, N. E. Statistical methods in cancer research. Vol I. The analysis of case-control studies. LARC Scientific Publication 32. IARC. Lyon,1980.

30. Correa-Cerro, L., Wohr, G., Haussler, J., Berthon, P., Drelon, E., Mangin, P., Fournier, G., Cussenot, O., Kraus, P., Just, W., Paiss, T., Cantu, J. M., Vogel, W. (CAG)nCAA and GGN repeats in the human androgen receptor gene are not associated with prostate cancer in a French-German population. Eur. J. Hum. Genet., 7: 357-362, 1999.

31. Wallen M J, Linja M, Kaartinen K, Schleutker J, Visakorpi T. Androgen receptor gene mutations in hormone-refractory prostate cancer. J. Pathol., 189: 559-563, 1999.

32. Koivisto, P. A., Rantala, I. Amplification of the androgen receptor gene is associated with P53 mutation in hormone-refractory recurrent prostate cancer. J. Pathol.,187: 237-241, 1999.

33. Culig, Z., Hobisch, A., Cronauer, M. V., Cato, A. C., Hittmair, A., Radmayr, C., Eberle, J., Bartsch, G., and Klocker, H. Mutant androgen receptor detected in an advanced-stage prostatic carcinoma is activated by adrenal androgens and progesterone. Mol. Endocrinol., 7: 1541-1550, 1997.

34. Schoenberg, M. P., Hakimi, J. M., Wang, S., Bova, G. S., Epstein, J. I., Pischbeck, K. H., Isaacs, W. B., Walsh, P. C., Barrack, E. R. Microsatellite mutation (CAG24→18) in the androgen receptor gene in human prostate cancer. Biochem. Biophys. Res. Commun.,198: 74-80, 1994.

35. Jenster, G. The role of the androgen receptor in the development and progression of prostate cancer. Semin. Oncol., 26: 407-21, 1999.

36. Koivisto, P., Kononen, J., Palmberg, C., Tammela, T., Hyytinen, E., Isola, J., Trapman, J., Cleutjens, K., Noordzij, A., Visakorpi, T., Kallioniemi, O. P. Androgen receptor gene amplification: a possible molecular mechanism for androgen deprivation therapy failure in prostate cancer. Cancer Res., 57: 314-319, 1997.

37. Culig, Z., Hobisch, A., Cronauer, M. V., Hittmair, A., Radmayr, C., Bartsch, G., and Kloclker, H. Activation of the androgen receptor by polypeptide growth factors and cellular regulators. World J. Urol., 13: 285-289, 1995.

38. McAbee, M. D., and Doncarlos, L. L. Estrogen, but not androgens, regulates androgen receptor messenger ribonucleic acid expression in the developing male rat forebrain. Endocrinology, 140: 3674-3681, 1999.

39. Gupta, C. Modulation of androgen receptor (AR)-mediated transcriptional activity by EGF in the developing mouse reproductive tract primary cells. Mol. Cell. Endocrinol., 152: 169-178, 1999.

40. Cude, K. J., Dixon, S. C., Guo, Y., Lisella, I., Figg, W. D. The androgen receptor: genetic considerations in the development and treatment of prostate cancer. J. Mol. Med., 77: 419-426, 1999.

41. Yeh, S., and Chang, C. Cloning and characterization of a specific coactivator, ARA70, for the androgen receptor in human prostate cells. Proc. Natl. Acad. Sci., 93: 5517-5521, 1997.

42. Yeh, S., Miyamoto, H., Shima, H., and Chang, C. From estrogen to androgen receptor: a new pathway for sex hormones in prostate. Proc. Natl. Acad. Sci., 95: 5527-5532, 1998.

43. Anzick, S. L., Kononen, J., Walker, R. L., Azorsa, D. O., Tanner, M. M., Guan, X. Y., Sauter, G., Kallioniemi, O. P., Trent, J. M., and Meltzer, P. S. AIB1, a steroid receptor coactivator amplified in breast and ovarian cancer. Science, 277: 965-968, 1997.

### H. SEQUENCES

| SEQ ID NO:1 first primer | | |
|---|---|---|
| 5′-TCA TCA CCT CCG ACA ACA GAG G-3′ | | |
| SEQ ID NO:2 second primer | | |
| 5′-TAT GGA AAC TGT TGC GGA GGA G-3′ | | |
| SEQ ID NO:3 complement to first primer | | |
| 5′-CCT CTG TTG TCG GAG GTG ATG A-3′ | | |
| SEQ ID NO:4 complement to second primer | | |
| 5′-CTC CTC CGC AAC AGT TTC CAT A-3′ | | |
| SEQ ID NO:5 general CAG/CAA sequence | | |
| 5′-(CAG)_{N} CAA (CAG)_{N} (CAAGAG)₄ CAG CAA CAG CAG CAA-3′ | | |
| Nucleotides 1-3 and 6-9 as a triplets must be at least one triple but can be any | | |
| multiple of triplets. | | |
| SEQ ID NO:6 one particular 29 mer sequence | | |
| 5′-CAG CAG CAG CAG CAG CAG CAA CAG CAG CAG CAG CAG CAG CAG | | |
| CAG CAG CAA CAG CAA CAG CAA CAG CAA CAG CAG CAA CAG CAG | | |
| CAA-3′ | | |
| SEQ ID NO:7 first androgen receptor CAG primer | | |
| 5′-GCT CTG GGA CGC AACCTCTCT-3′ | | |
| SEQ ID NO:8 second androgen receptor CAG primer | | |
| 5′-GCA GCG ACT ACC GCA TCA TCA-3′ | | |
| SEQ ID NO:9 complement to first androgen receptor CAG primer | | |
| 5′-AGAGAGGTTGCGTCCCAGAGC-3′ | | |
| SEQ ID NO:10 complement to second androgen receptor CAG primer | | |
| 5′-TGATGATGCGGTAGTCGCTGC-3′ | | |
| SEQ ID NO:11 first androgen receptor GGN primer | | |
| 5′ACCCTCAGCCGCCGCTTCCTCATC-3′ | | |
| SEQ ID NO:12 second androgen receptor GGN primer | | |
| 5′-CTGGGATAGGGCACTCTGCTCAAC-3′ | | |
| SEQ ID NO:13 complement to first androgen receptor GGN primer | | |
| 5′-GATGAGGAAGCGGCGGCTGAGGGT-3′ | | |
| SEQ ID NO:14 complement to second androgen receptor GGN primer | | |
| 5′-GTTGAGCAGAGTGCCCTATCCCAG-3′ | | |
| SEQ ID NO:15 | | |
| 5′-CGGG-GTAAGGGAAGTAGGTGGAAG-3′ | | |
| SEQ ID NO:16 | | |
| and 5′-CTCTACGATGGGCTTGGGGAGAAC-3′ | | |
| SEQ ID NO:17 | | |
| GGT₃GGG₁GGT₂GGCₙ | | |
| | SEQ ID NO:18 AIB1 sequence Genbank accession number XM_030032 | |
| | 1 | cggcagcggc tgcggcttag tcggtggcgg ccggcggcgg ctgcgggctg agcggcgagt |
| | 61 | ttccgattta aagctgagct gcgaggaaaa tggcggcggg aggatcaaaa tacttgctgg |
| | 121 | atggtggact cagagaccaa taaaaataaa ctgcttgaac atcctttgac tggttagcca |
| | 181 | gttgctgatg tatattcaag atgagtggat taggagaaaa cttggatcca ctggccagtg |
| | 241 | attcacgaaa acgcaaattg ccatgtgata ctccaggaca aggtcttacc tgcagtggtg |
| | 301 | aaaaacggag acgggagcag gaaagtaaat atattgaaga attggctgag ctgatatctg |
| | 361 | ccaatcttag tgatattgac aatttcaatg tcaaaccaga taaatgtgcg attttaaagg |
| | 421 | aaacagtaag acagatacgt caaataaaag agcaaggaaa aactatttcc aatgatgatg |
| | 481 | atgttcaaaa agccgatgta tcttctacag ggcagggagt tattgataaa gactccttag |
| | 541 | gaccgctttt acttcaggca ttggatggtt tcctatttgt ggtgaatcga gacggaaaca |
| | 601 | ttgtatttgt atcagaaaat gtcacacaat acctgcaata taagcaagag gacctggtta |
| | 661 | acacaagtgt ttacaatatc ttacatgaag aagacagaaa ggattttctt aagaatttac |
| | 721 | caaaatctac agttaatgga gtttcctgga caaatgagac ccaaagacaa aaaagccata |
| | 781 | catttaattg ccgtatgttg atgaaaacac cacatgatat tctggaagac ataaacgcca |
| | 841 | gtcctgaaat gcgccagaga tatgaaacaa tgcagtgctt tgccctgtct cagccacgag |
| | 901 | ctatgatgga ggaaggggaa gatttgcaat cttgtatgat ctgtgtggca cgccgcatta |
| | 961 | ctacaggaga aagaacattt ccatcaaacc ctgagagctt tattaccaga catgatcttt |
| | 1021 | caggaaaggt tgtcaatata gatacaaatt cactgagatc ctccatgagg cctggctttg |
| | 1081 | aagatataat ccgaaggtgt attcagagat tttttagtct aaatgatggg cagtcatggt |
| | 1141 | cccagaaacg tcactatcaa gaagcttatc ttaatggcca tgcagaaacc ccagtatatc |
| | 1201 | gattctcgtt ggctgatgga actatagtga ctgcacagac aaaaagcaaa ctcttccgaa |
| | 1261 | atcctgtaac aaatgatcga catggctttg tctcaaccca cttccttcag agagaacaga |
| | 1321 | atggatatag accaaaccca aatcctgttg gacaagggat tagaccacct atggctggat |
| | 1381 | gcaacagttc ggtaggcggc atgagtatgt cgccaaacca aggcttacag atgccgagca |
| | 1441 | gcagggccta tggcttggca gaccctagca ccacagggca gatgagtgga gctaggtatg |
| | 1501 | ggggttccag taacatagct tcattgaccc ctgggccagg catgcaatca ccatcttcct |
| | 1561 | accagaacaa caactatggg ctcaacatga gtagcccccc acatgggagt cctggtcttg |
| | 1621 | ccccaaacca gcagaatatc atgatttctc ctcgtaatcg tgggagtcca aagatagcct |
| | 1681 | cacatcagtt ttctcctgtt gcaggtgtgc actctcccat ggcatcttct ggcaatactg |
| | 1741 | ggaaccacag cttttccagc agctctctca gtgccctgca agccatcagt gaaggtgtgg |
| | 1801 | ggacttccct tttatctact ctgtcatcac caggccccaa attggataac tctcccaata |
| | 1861 | tgaatattac ccaaccaagt aaagtaagca atcaggattc caagagtcct ctgggctttt |
| | 1921 | attgcgacca aaatccagtg gagagttcaa tgtgtcagtc aaatagcaga gatcacctca |
| | 1981 | gtgacaaaga aagtaaggag agcagtgttg agggggcaga gaatcaaagg ggtcctttgg |
| | 2041 | aaagcaaagg tcataaaaaa ttactgcagt tacttacctg ttcttctgat gaccggggtc |
| | 2101 | attcctcctt gaccaactcc cccctagatt caagttgtaa agaatcttct gttagtgtca |
| | 2161 | ccagcccctc tggagtctcc tcctctacat ctggaggagt atcctctaca tccaatatgc |
| | 2221 | atgggtcact gttacaagag aagcaccgga ttttgcacaa gttgctgcag aatgggaatt |
| | 2281 | caccagctga ggtagccaag attactgcag aagccactgg gaaagacacc agcagtataa |
| | 2341 | cttcttgtgg ggacggaaat gttgtcaagc aggagcagct aagtcctaag aagaaggaga |
| | 2401 | ataatgcact tcttagatac ctgctggaca gggatgatcc tagtgatgca ctctctaaag |
| | 2461 | aactacagcc ccaagtggaa ggagtggata ataaaatgag tcagtgcacc agctccacca |
| | 2521 | ttcctagctc aagtcaagag aaagacccta aaattaagac agagacaagt gaagagggat |
| | 2581 | ctggagactt ggataatcta gatgctattc ttggtgatct gactagttct gacttttaca |
| | 2641 | ataattccat atcctcaaat ggtagtcatc tggggactaa gcaacaggtg tttcaaggaa |
| | 2701 | ctaattctct gggtttgaaa agttcacagt ctgtgcagtc tattcgtcct ccatataacc |
| | 2761 | gagcagtgtc tctggatagc cctgtttctg ttggctcaag tcctccagta aaaaatatca |
| | 2821 | gtgctttccc catgttacca aagcaaccca tgttgggtgg gaatccaaga atgatggata |
| | 2881 | gtcaggaaaa ttatggctca agtatgggtg ggccaaaccg aaatgtgact gtgactcaga |
| | 2941 | ctccttcctc aggagactgg ggcttaccaa actcaaaggc cggcagaatg gaacctatga |
| | 3001 | attcaaactc catgggaaga ecaggaggag attataatac ttctttaccc agacctgcac |
| | 3061 | tgggtggctc tattcccaca ttgcctcttc ggtctaatag cataccaggt gogagaccag |
| | 3121 | tattgcaaca gcagcagcag atgcttcaaa tgaggcctgg tgaaatcccc atgggaatgg |
| | 3181 | gggctaatcc ctatggccaa gcagcagcat ctaaccaact gggttcctgg cccgatggca |
| | 3241 | tgttgtccat ggaacaagtt tctcatggca ctcaaaatag gcctcttctt aggaattccc |
| | 3301 | tggatgatct tgttgggcca ccttccaacc tggaaggcca gagtgacgaa agageattat |
| | 3361 | tggaccagct gcacactctt ctcagcaaca cagatgccac aggcctggaa gaaattgaca |
| | 3421 | gagctttggg cattcctgaa cttgtcaatc agggacaggc attagagccc aaacaggatg |
| | 3481 | ctttccaagg ccaagaagca gcagtaatga tggatcagaa ggcaggatta tatggacaga |
| | 3541 | catacccagc acaggggcct ccaatgcaag gaggctttca tcttcaggga caatcaccat |
| | 3601 | cttttaactc tatgatgaat cagatgaacc agcaaggcaa ttttcctctc caaggaatgc |
| | 3661 | acccacgagc caacatcatg agaccccgga caaacacccc caagcaactt agaatgcagc |
| | 3721 | ttcagcagag gctgcagggc cagcagtttt tgaatcagag ccgacaggca cttgaattga |
| | 3781 | aaatggaaaa ccctactgct ggtggtgctg cggtgatgag gcctatgatg cagccccagc |
| | 3841 | agggttttct taatgctcaa atggtcgccc aacgcagcag agagctgcta agtcatcact |
| | 3901 | tccgacaaca gagggtggct atgatgatgc agcagcagca gcagcagcaa cagcagcagc |
| | 3961 | agcagcagca gcagcagcaa cagcaacagc aacagcaaca gcagcaacag cagcaaaccc |
| | 4021 | aggccttcag cccacctcct aatgtgactg cttcccccag catggatggg cttttggcag |
| | 4081 | gacccacaat gccacaagct cctccgcaac agtttccata tcaaccaaat tatggaatgg |
| | 4141 | gacaacaacc agatccagcc tttggtcgag tgtctagtcc tcccaatgca atgatgtcgt |
| | 4201 | caagaatggg tccctcccag aatcccatga tgcaacaccc gcaggctgca tccatctatc |
| | 4261 | agtcctcaga aatgaagggc tggccatcag gaaatttggc caggaacagc tccttttccc |
| | 4321 | agcagcagtt tgcccaccag gggaatcctg cagtgtatag tatggtgcac atgaatggca |
| | 4381 | gcagtggtca catgggacag atgaacatga accccatgcc catgtctggc atgcctatgg |
| | 4441 | gtcctgatca gaatactgct gacatctctg caccaggacc tcttaaggaa accactgtac |
| | 4501 | aaatgacact gcactaggat tattgggaag gaatcattgt tccaggcatc catcttggaa |
| | 4561 | gaaaggacca gctttgagct ccatcaaggg tattttaagt gatgtcattt gagcaggact |
| | 4621 | ggattttaag ccgaagggca atatctacgt gtttttcccc cctccttctg ctgtgtatca |
| | 4681 | tggtgttcaa aacagaaatg ttttttggca ttccacctcc tagggatata attctggaga |
| | 4741 | catggagtgt tactgatcat aaaacttttg tgtcactttt ttctgccttg ctagccaaaa |
| | 4801 | tctcttaaat acacgtaggt gggccagaga acattggaag aatcaagaga gattagaata |
| | 4861 | tctggtttct ctagttgcag tattggacaa agagcatagt cccagccttc aggtgtagta |
| | 4921 | gttctgtgtt gaccctttgt ccagtggaat tggtgattct gaattgtcct ttactaatgg |
| | 4981 | tgttgagttg ctctgtccct attatttgcc ctaggctttc tcctaatgaa ggttttcatt |
| | 5041 | tgccattcat gtcctgtaat acttcacctc caggaactgt catggatgtc caaatggctt |
| | 5101 | tgcagaaagg aaatgagatg acagtattta atcgcagcag tagcaaactt ttcacatgct |
| | 5161 | aatgtgcagc tgagtgcact ttatttaaaa agaatggata aatgcaatat tcttgaggtc |
| | 5221 | ttgagggaat agtgaaacac attcctggtt tttgcctaca cttacgtgtt agacaagaac |
| | 5281 | tatgattttt ttttttaaag tactggtgtc accctttgcc tatatggtag agcaataatg |
| | 5341 | ctttttaaaa ataaacttct gaaaacccaa ggccaggtac tgcattctga atcagaatct |
| | 5401 | cgcagtgttt ctgtgaatag atttttttgt aaatatgacc tttaagatat tgtattatgt |
| | 5461 | aaaatatgta tatacctttt tttgtaggtc acaacaactc atttttacag agtttgtgaa |
| | 5521 | gctaaatatt taacattgtt gatttcagta agctgtgtgg tgaggctacc agtggaagag |
| | 5581 | acatcccttg acttttgtgg cctgggggag gggtagtgct ccacagcttt tccttcccca |
| | 5641 | ccccccagcc ttagatgcct cgctcttttc aatctcttaa tctaaatgct ttttaaagag |
| | 5701 | attatttgtt tagatgtagg cattttaatt ttttaaaaat tcctctacca gaactaagca |
| | 5761 | ctttgttaat ttggggggaa agaatagata tggggaaata aacttaaaaa aaaatcagga |
| | 5821 | atttaaaaaa acgagcaatt tgaagagaat cttttggatt ttaagcagtc cgaaataata |
| | 5881 | gcaattcatg ggctgtgtgt gtgtgtgtat gtgtgtgtgt gtgtgtgtat gtttaattat |
| | 5941 | gttacctttt catccccttt aggagcgttt tcagattttg gttgctaaga cctgaatccc |
| | 6001 | atattgagat ctcgagtaga atccttggtg tggtttctgg tgtctgctca gctgtcccct |
| | 6061 | cattctacta atgtgatgct ttcattatgt ccctgtggat tagaatagtg tcagttattt |
| | 6121 | cttaagtaac tcagtaccca gaacagccag ttttactgtg attcagagcc acagtctaac |
| | 6181 | tgagcacctt ttaaacccct ccctcttctg ccccctacca cttttctgct gttgcctctc |
| | 6241 | tttgacacct gttttagtca gttgggagga agggaaaaat caagtttaat tccctttatc |
| | 6301 | tgggttaatt catttggttc aaatagttga cggaattggg tttctgaatg tctgtgaatt |
| | 6361 | tcagaggtct ctgctagcct tggtatcatt ttctagcaat aactgagagc cagttaattt |
| | 6421 | taagaatttc acacatttag ccaatctttc tagatgtctc tgaaggtaag atcatttaat |
| | 6481 | atctttgata tgcttacgag taagtgaatc ctgattattt ccagacccac caccagagtg |
| | 6541 | gatcttattt tcaaagcagt atagacaatt atgagtttgc cctctttccc ctaccaagtt |
| | 6601 | caaaatatat ctaagaaaga ttgtaaatcc gaaaacttcc attgtagtgg cctgtgcttt |
| | 6661 | tcagatagta tactctcctg tttggagaca gaggaagaac caggtcagtc tgtctctttt |
| | 6721 | tcagctcaat tgtatctgac ccttctttaa gttatgtgtg tggggagaaa tagaatggtg |
| | 6781 | ctcttatctt tcttgacttt aaaaaaatta ttaaaaacaa aaaaaaaata aa |
| | | SEQ ID NO:19 |
| | | MSGLGENLDPLASDSRRKIYCDTPGQGLTCSGEKRRREQESKY |
| | | IEELAELISANLSDIDNFNVKPDKCAILKETVRQIRQIKEQGKTISNDDDVQKADVSS |
| | | TGQGVDKDSLGPLLLQAIDGFLFVVNRGNWFVSENVTQYLQYKQEDLVNTSVYNI |
| | | LHEEDRKDFLKNLPKSTVNGVSWTNETQRQKSHTFNCRMLMKTPHDILEDINASPEMR |
| | | QRYETMQCFALSQPRAMMEEGEDLQSCMICVARRITTGERTFPSNPESFITRHDLSGK |
| | | VVNIDTNSLRSSMRPGFEDIIRRCIQRFFSLNDGQSWSQKRHYQEAYLNGHAETPVYR |
| | | FSLADGTIVTAQTKSKLFRNPVTNDRHGFVSTHFLQREQNGYRPNPNPVGQGIRPPMA |
| | | GCNSSVGGMSMSPNQGLQMYSSRAYGLADPSTTGQMSGARYGGSSNIASLTPGPGMQS |
| | | PSSYQNNNYGLNMSSPPHGSPGLAPNQQNIMISPRNRGSPKIASHQFSPVAGVHSPMA |
| | | SSGNTGNHSPSSSSLSALQAISEGVGTSLLSTLSSPGPKLDNSPNMMTQPSKVSNQD |
| | | SKSPLGFYCDQNPVESSMCQSNSRDHLSDKESKESSVEGAENQRGPLESKGHKKLLQL |
| | | LTCSSDDRGHSSLTNSPLDSSCKESSVSVTSPSGVSSSTSGGVSSTSNMHGSLLQEKH |
| | | RILHKLLQNGNSPAEVAKITAEATGKDTSSITSCGDGNVVKQEQLSPKKKENNALLRY |
| | | LLDRDDPSDALSKELQPQVEGVDNKMSQCTSSTIPSSSQEKDPKIKTETSEEGSGDLD |
| | | NLDAILGDLTSSDFYNNSISSNGSHLGTKQQVFQGTNSLGLKSSQSVQSIRPPYNRAV |
| | | SLDSPVSVGSSPPVKNISAFPMLPKQPMLGGNPRMMDSQENYGSSMGGPNRNVTVTQT |
| | | PSSGDWGLPNSKAGRMEPMNSNSMGRPGGDYNTSLPRPALGGSIPTLPLRSNSIPGAR |
| | | PVLQQQQQMLQMRPGEIPMGMGANPYGQAAASNQLGSWPDGMLSMEQVSHGTQNRPLL |
| | | RNSLDDLVGPPSNLEGQSDERALLDQLHTLLSNTDATGLEEIDRALGIPELVNQGQAL |
| | | EPKQDAFQGQEAAVMMDQKAGLYGQTYPAQGPPMGGGFHLQGQSPSFNSMMNQMNQQG |
| | | NFPLQGMHPRANIMRPRTNTPKQLRMQLQQRLQGQQFLNQSRQALELKMENPTAGGAA |
| | | VMRPMMQPQQGFLNAQMVAQRSRELLSHHFRQQRVAMMMQQQQQQQQQQQQQQQQQQ |
| | | Q |
| | | QQQQQQQQQQTQAFSPPPNVTASPSMDGLLAGPTMYQAPPQQFPYQPNYGMGQQPDPA |
| | | FGRVSSPPNAMMSSRMGPSQNPMMQHPQAASIYQSSEMKGWPSGNLARNSSFSQQQFA |
| | | HQGNPAVYSMVHMNGSSGHMGQMNMNPMPMSGMPMGPDQNTADISAPGPLKETTVQMT |
| | | LH |
| | | SEQ ID NO:20 |
| | | 1:NM_000044 |
| | 1 | cgagatcccg gggagccagc ttgctgggag agcgggacgg tccggagcaa gcccacaggc |
| | 61 | agaggaggcg acagagggaa aaagggccga gctagccgct ccagtgctgt acaggagccg |
| | 121 | aagggacgca ccacgccagc cccagcccgg ctccagcgac agccaacgcc tcttgcagcg |
| | 181 | cggcggcttc gaagccgccg cccggagctg ccctttcctc ttcggtgaag tttttaaaag |
| | 241 | ctgctaaaga ctcggaggaa gcaaggaaag tgcctggtag gactgacggc tgcctttgtc |
| | 301 | ctcctcctct ccaccccgcc tccccccacc ctgccttccc cccctccccc gtcttctctc |
| | 361 | ccgcagctgc ctcagtcggc tactctcagc caacccccct caccaccctt ctccccaccc |
| | 421 | gcccccccgc ccccgtcggc ccagcgctgc cagcccgagt ttgcagagag gtaactccct |
| | 481 | ttggctgcga gcgggcgagc tagctgcaca ttgcaaagaa ggctcttagg agccaggcga |
| | 541 | ctggggagcg gcttcagcac tgcagccacg acccgcctgg ttagaattcc ggcggagaga |
| | 601 | accctctgtt ttcccccact ctctctccac ctcctcctgc cttccccacc ccgagtgcgg |
| | 661 | agcagagatc aaaagatgaa aaggcagtca ggtcttcagt agccaaaaaa caaaacaaac |
| | 721 | aaaaacaaaa aagccgaaat aaaagaaaaa gataataact cagttcttat ttgcacctac |
| | 781 | ttcagtggac actgaatttg gaaggtggag gattttgttt ttttctttta agatctgggc |
| | 841 | atcttttgaa tctacccttc aagtattaag agacagactg tgagcctagc agggcagatc |
| | 901 | ttgtccaccg tgtgtcttct tctgcacgag actttgaggc tgtcagagcg ctttttgcgt |
| | 961 | ggttgctccc gcaagtttcc ttctctggag cttcccgcag gtgggcagct agctgcagcg |
| | 1021 | actaccgcat catcacagcc tgttgaactc ttctgagcaa gagaagggga ggcggggtaa |
| | 1081 | gggaagtagg tggaagattc agccaagctc aaggatggaa gtgcagttag ggctgggaag |
| | 1141 | ggtctaccct cggccgccgt ccaagaccta ccgaggagct ttccagaatc tgttccagag |
| | 1201 | cgtgcgcgaa gtgatccaga acccgggccc caggcaccca gaggccgcga gcgcagcacc |
| | 1261 | tcccggcgcc agtttgctgc tgctgcagca gcagcagcag cagcagcagc agcagcagca |
| | 1321 | gcagcagcag cagcagcagc agcagcaaga gactagcccc aggcagcagc agcagcagca |
| | 1381 | gggtgaggat ggttctcccc aagcccatcg tagaggcccc acaggctacc tggtcctgga |
| | 1441 | tgaggaacag caaccttcac agccgcagtc ggccctggag tgccaccccg agagaggttg |
| | 1501 | cgtcccagag cctggagccg ccgtggccgc cagcaagggg ctgccgcagc agctgccagc |
| | 1561 | acctccggac gaggatgact cagctgcccc atccacgttg tccctgctgg gccccacttt |
| | 1621 | ccccggctta agcagctgct ccgctgacct taaagacatc ctgagcgagg ccagcaccat |
| | 1681 | gcaactcctt cagcaacagc agcaggaagc agtatccgaa ggcagcagca gcgggagagc |
| | 1741 | gagggaggcc tcgggggctc ccacttcctc caaggacaat tacttagggg gcacttcgac |
| | 1801 | catttctgac aacgccaagg agttgtgtaa ggcagtgtcg gtgtccatgg gcctgggtgt |
| | 1861 | ggaggcgttg gagcatctga gtccagggga acagcttcgg ggggattgca tgtacgcccc |
| | 1921 | acttttggga gttccacccg ctgtgcgtcc cactccttgt gccccattgg ccgaatgcaa |
| | 1981 | aggttctctg ctagacgaca gcgcaggcaa gagcactgaa gatactgctg agtattcccc |
| | 2041 | tttcaaggga ggttacacca aagggctaga aggcgagagc ctaggctgct ctggcagcgc |
| | 2101 | tgcagcaggg agctccggga cacttgaact gccgtctacc ctgtctctct acaagtccgg |
| | 2161 | agcactggac gaggcagctg cgtaccagag tcgcgactac tacaactttc cactggctct |
| | 2221 | ggccggaccg ccgccccctc cgccgcctcc ccatccccac gctcgcatca agctggagaa |
| | 2281 | cccgctggac tacggcagcg cctgggcggc tgcggcggcg cagtgccgct atggggacct |
| | 2341 | ggcgagcctg catggcgcgg gtgcagcggg acccggttct gggtcaccct cagccgccgc |
| | 2401 | ttcctcatcc tggcacactc tcttcacagc cgaagaaggc cagttgtatg gaccgtgtgg |
| | 2461 | tggtggtggg ggtggtggcg gcggcggcgg cggcggcggc ggcggcggcg gcggcggcgg |
| | 2521 | cggcggcggc gaggcgggag ctgtagcccc ctacggctac actcggcccc ctcaggggct |
| | 2581 | ggcgggccag gaaagcgact tcaccgcacc tgatgtgtgg taccctggcg gcatggtgag |
| | 2641 | cagagtgccc tatcccagtc ccacttgtgt caaaagcgaa atgggcccct ggatggatag |
| | 2701 | ctactccgga ccttacgggg acatgcgttt ggagactgcc agggaccatg ttttgcccat |
| | 2761 | tgactattac tttccacccc agaagacctg cctgatctgt ggagatgaag cttctgggtg |
| | 2821 | tcactatgga gctctcacat gtggaagctg caaggtcttc ttcaaaagag ccgctgaagg |
| | 2881 | gaaacagaag tacctgtgcg ccagcagaaa tgattgcact attgataaat tccgaaggaa |
| | 2941 | aaattgtcca tcttgtcgtc ttcggaaatg ttatgaagca gggatgactc tgggagcccg |
| | 3001 | gaagctgaag aaacttggta atctgaaact acaggaggaa ggagaggctt ccagcaccac |
| | 3061 | cagccccact gaggagacaa cccagaagct gacagtgtca cacattgaag gctatgaatg |
| | 3121 | tcagcccatc tttctgaatg tcctggaagc cattgagcca ggtgtagtgt gtgctggaca |
| | 3181 | cgacaacaac cagcccgact cctttgcagc cttgctctct agcctcaatg aactgggaga |
| | 3241 | gagacagctt gtacacgtgg tcaagtgggc caaggccttg cctggcttcc gcaacttaca |
| | 3301 | cgtggacgac cagatggctg tcattcagta ctcctggatg gggctcatgg tgtttgccat |
| | 3361 | gggctggcga tccttcacca atgtcaactc caggatgctc tacttcgccc ctgatctggt |
| | 3421 | tttcaatgag taccgcatgc acaagtcccg gatgtacagc cagtgtgtcc gaatgaggca |
| | 3481 | cctctctcaa gagtttggat ggctccaaat caccccccag gaattcctgt gcatgaaagc |
| | 3541 | actgctactc ttcagcatta ttccagtgga tgggctgaaa aatcaaaaat tctttgatga |
| | 3601 | acttcgaatg aactacatca aggaactcga tcgtatcatt gcatgcaaaa gaaaaaatcc |
| | 3661 | cacatcctgc tcaagacgct tctaccagct caccaagctc ctggactccg tgcagcctat |
| | 3721 | tgcgagagag ctgcatcagt tcacttttga cctgctaatc aagtcacaca tggtgagcgt |
| | 3781 | ggactttccg gaaatgatgg cagagatcat ctctgtgcaa gtgcccaaga tcctttctgg |
| | 3841 | gaaagtcaag cccatctatt tccacaccca gtgaagcatt ggaaacccta tttccccacc |
| | 3901 | ccagctcatg ccccctttca gatgtcttct gcctgttata actctgcact actcctctgc |
| | 3961 | agtgccttgg ggaatttcct ctattgatgt acagtctgtc atgaacatgt tcctgaattc |
| | 4021 | tatttgctgg gctttttttt tctctttctc tcctttcttt ttcttcttcc ctccctatct |
| | 4081 | aaccctccca tggcaccttc agactttgct tcccattgtg gctcctatct gtgttttgaa |
| | 4141 | tggtgttgta tgcctttaaa tctgtgatga tcctcatatg gcccagtgtc aagttgtgct |
| | 4201 | tgtttacagc actactctgt gccagccaca caaacgttta cttatcttat gccacgggaa |
| | 4261 | gtttagagag ctaagattat ctggggaaat caaaacaaaa aacaagcaaa caaaaaaaaa |
| | 4321 | a |

SEQ ID NO:21
1:NM_000044
MEVQLGLGRVYPRPPSKTYRGAFQNLFQSVREVIQNPGPRHPEA
ASAAPPGASLLLLQQQQQQQQQQQQQQQQQQQQQETSPRQQQQQQGEDGSPQAHRRGP
TGYLVLDEEQQPSQPQSALECHPERGCVPEPGAAVAASKGLPQQLPAPPDEDDSAAPS
TLSLLGPIFPGLSSCSADLKDILSEASTMQLLQQQQQEAVSEGSSSGRAREASGAPTS
SKDNYLGGTSTISDNAKELCKAVSVSMGLGVEALEHLSPGEQLRGDCMYAPLLGVPPA
VRPTPCAPLAECKGSLLDDSAGKSTEDTAEYSPFKGGYTKGLEGESLGCSGSAAAGSS
GTLELPSTLSLYKSGALDEAAAYQSRDYYNFPLALAGPPPPPPPPPHARIKLENPLD
YGSAWAAAAQCRYGDLASLHGAGAAGPGSGSPSAAASSSWHTLFTAEEGQLYGPCGG
GGGGGGGGGGGGGGGGGGGGGGEAGAVAPYGYTRPPQGLAGQESDFTAPDVWYPGGMV
SRVPYPSPTCVKSEMGPWMDSYSGPYGDMRLETARDHVLPIDYYFPPQKTCLICGDEA
SGCHYGALTCGSCKVFFKRAAEGKQKYLCASRNDCTIDKFRRKNCPSCRLRKCYEAGM
TLGARKLKKLGNLKLQEEGEASSTTSPTEETTQKLTVSHIEGYECQPIFLNVLEAIEP
GVVCAGHDNNQPDSFAALLSSLNELGERQLVHVVKWAKALPGFRNLHVDDQMAVIQYS
WMGLMVFAMGWRSFTNVNSRMLYFAPDLVFNEYRMHKSRMYSQCVRMRHLSQEFGWLQ
ITPQEFLCMKALLLFSIIPVDGLKNQKFFDELRMNYIKELDRIIACKRKMPTSCSRRF
YQLTKLLDSVQPIARELHQFTFDLLIKSHMVSVDFPEMMAEIISVQVPKILSGKVKPI
YFHTQ

What is claimed is:

1. A method for assessing the risk of prostate cancer in a human subject comprising determining the length of the contiguous CAG or CAA repeats in both AIB1 gene alleles of the subject and assessing whether the length of the CAG or CAA repeats is less than, equal to, or greater than 29 repeats, a length less than or greater than 29 repeats in both alleles indicating an increased risk of prostate cancer in the subject.

2. The method of claim 1, wherein determining the length of the repeats comprises amplifying a region of both AIB1 gene alleles comprising the contiguous CAG or CAA repeat.

3. The method of claim 2, wherein the amplification is by PCR that produces two PCR products.

4. The method of claim 3, further comprising analyzing the PCR products by chromatography.

5. The method of claim 4, wherein the chromatography is gel electrophoresis.

6. The method of claim 2, wherein the sequence of the PCR products is determined.

7. The method of claim 3, wherein the PCR product is produced using a first AIB1 primer that selectively hybridizes with the sequence set forth in SEQ ID NO:3 and a second AIB1 primer that selectively hybridizes with the sequence set forth in SEQ ID NO:4.

8. The method of claim 7, wherein the first AIB1 primer has the sequence set forth in SEQ ]D NO:1 and the second AIB1 primer has the sequence set forth in SEQ ID NO:2.

9. The method of claim 1, wherein determining how many CAG or CAA repeats there are comprises sequencing the CAG or CAA repeats.

10. A method for assessing the risk of prostate cancer in a human subject comprising determining the length of the contiguous CAG or CAA repeats in the AIB1 gene alleles of the subject and assessing whether the length of the CAG or CAA repeats in each allele is less than, equal to, or greater than 29 repeats, and determining the length of the contiguous CAG or CAA repeats in the androgen receptor gene of the subject and assessing whether the length of the CAG or CAA repeats is less than, equal to, or greater than 23 repeats, a length in at least one allele less than or greater than 29 repeats in the AIBL gene and less than or greater than 23 repeats in the androgen receptor gene indicating an increased risk of prostate cancer in the subject.

11. The method of claim 10, wherein determining the length of the contiguous CAG or CAA repeats in the AIB1 gene alleles comprises amplifying a region of the AIB1 gene alleles comprising the CAG or CAA repeats and wherein determining the length of the CAG or CAA repeats in the androgen receptor gene comprises amplifying a region of the androgen receptor gene comprising the CAG or CAA repeats.

12. The method of claim 11, wherein the amplification of the regions of the AIB1 gene and the androgen receptor gene is by PCR that produces a first and a second AIB1 PCR product and an androgen receptor PCR product.

13. The method of claim 12, further comprising analyzing the PCR products by chromatography.

14. The method of claim 13, wherein the chromatography is gel electrophoresis.

15. The method of claim 12, wherein the sequence of the PCR products is determined.

16. The method of claim 12, wherein the AIB1 PCR product is produced using a first AIB1 primer that selectively hybridizes with the sequence set forth in SEQ ID NO:3 and a second AIB1 primer that selectively hybridizes with the sequence set forth in SEQ ID NO:4.

17. The method of claim 16, wherein the first AIB1 primer has the sequence set forth in SEQ ID NO:1 and the second AIBI primer has the sequence set forth in SEQ ID NO:2.

18. The method of claim 12, wherein the androgen receptor PCR product is produced using a first androgen receptor CAG primer that selectively hybridizes with the sequence set forth in SEQ ID NO:9 and a second androgen receptor CAG primer that selectively hybridizes with the sequence set forth in SEQ ID NO:10.

19. The method of claim 1S, wherein the first androgen receptor CAG primer has the sequence set forth in SEQ ID NO:7 and the second androgen receptor CAG primer has the sequence set forth in SEQ ID NO:8.

20. The method of claim 12, wherein the AIB1 PCR product is produced using a first AMBI primer that selectively hybridizes with the sequence set forth in SEQ ID NO:3 and a second AIB1 primer that selectively hybridizes with the sequence set forth in SEQ ID NO:4. and wherein the androgen receptor PCR product is produced using a first androgen receptor CAG primer that selectively hybridizes with the sequence set forth in SEQ ID NO:9 and a second androgen receptor CAG primer that selectively hybridizes with the sequence set forth in SEQ ID NO:10.

21. The method of claim 20, wherein the first AIB1 primer has the sequence set forth in SEQ ID NO:1 and the second AIB1 primer has the sequence set forth in SEQ ID NO:2 and wherein the first androgen receptor CAG primer has the sequence set forth in SEQ ID NO:7 and the second androgen receptor CAG primer has the sequence set forth in SEQ ID NO:8.

22. The method of claim 10, wherein more than 29 contiguous CAG or CAA repeats in at least one allele of the AIB1 gene of the person and more than 23 contiguous CAG or CAA repeats in the androgen receptor gene of the person indicates an increased risk of prostate cancer in the subject.

23. The method of claim 10, wherein more than 29 contiguous CAG or CAA repeats in at least one allele of the AIB1 gene of the person and less than 23 contiguous CAG or CAA repeats in the androgen receptor gene of the person indicates an increased risk of prostate cancer in the subject.

24. The method of claim 10, wherein less than 29 contiguous CAG or CAA repeats in at least one allele of the AIB1 gene of the person and more than 23 contiguous CAG or CAA repeats in the androgen receptor gene of the person indicates an increased risk of prostate cancer in the subject.

25. The method of claim 10, wherein less than 29 contiguous CAG or CAA repeats in at least one allele of the AIB1 gene of the person and less than 23 contiguous CAG or CAA repeats in the androgen receptor gene of the person indicates an increased risk of prostate cancer in the subject.

26. The method of claim 10, wherein more or less than 29 contiguous CAG or CAA repeats in both alleles of the AIB1 gene of the person and more than 23 contiguous CAG or CAA repeats in the androgen receptor gene of the person indicates an increased risk of prostate cancer in the subject.

27. The method of claim 10, wherein more or less than 29 contiguous CAG or CAA repeats in both alleles of the AIB1 gene of the person and less than 23 contiguous CAG or CAA repeats in the androgen receptor gene of the person indicates an increased risk of prostate cancer in the subject.

28. The method of claim 10, wherein more than 29 contiguous CAG or CAA repeats in both alleles of the AIB1 gene of the person and more than 23 contiguous CAG or CAA repeats in the androgen receptor gene of the person indicates an increased risk of prostate cancer in the subject.

29. The method of claim 10, wherein less than 29 contiguous CAG or CAA repeats in both alleles of the AIB1 gene of the person and less than 23 contiguous CAG or CAA repeats in the androgen receptor gene of the person indicates an increased risk of prostate cancer in the subject.

30. A method for assessing the risk of prostate cancer in a human subject comprising determining the length of the contiguous CAG or CAA repeats in the AIB1 gene alleles of the subject and assessing whether the length of the CAG or CAA repeats in each allele is less than, equal to, or greater than 29 repeats, and determining the length of the contiguous GGN repeats in the androgen receptor gene of the subject and assessing whether the length of the GGN repeats is less than, equal to, or greater than 23 repeats, a length in at least one allele less than or greater than 29 repeats in the AIB1 gene and less than or greater than 23 repeats in the androgen receptor gene indicating an increased risk of prostate cancer in the subject, wherein N is either T, G, or C.

31. The method of claim 30, wherein determining the length of the contiguous CAG or CAA repeats in the AIB1 gene alleles comprises amplifying a region of the AIB1 gene alleles comprising the CAG or CAA repeats and wherein determining the length of the contiguous GGN repeats in the androgen receptor gene comprises amplifying a region of the androgen receptor gene comprising the GGN repeats.

32. The method of claim 31, wherein the amplification of the regions of the AIB1 gene and the androgen receptor gene is by PCR that produces a first and a second AIB1 PCR product and an androgen receptor PCR product.

33. The method of claim 32, further comprising analyzing the PCR products by chromatography.

34. The method of claim 33, wherein the chromatography is gel electrophoresis.

35. The method of claim 32, wherein the sequence of the PCR products is determined.

36. The method of claim 32, wherein the AIB1 PCR product is produced using a first AIB1 primer that selectively hybridizes with the sequence set forth in SEQ ID NO:3 and a second AIB1 primer that selectively hybridizes with the sequence set forth in SEQ ID NO:4.

37. The method of claim 36, wherein the first AIB1 primer has the sequence set forth in SEQ ID NO:1 and the second AIB1 primer has the sequence set forth in SEQ ID NO:2.

38. The method of claim 32, wherein the androgen receptor PCR product is produced using a first androgen receptor GGN primer that selectively hybridizes with the sequence set forth in SEQ ID NO:13 and a second androgen receptor GGN primer that selectively hybridizes with the sequence set forth in SEQ ID NO:14.

39. The method of claim 38, wherein the first androgen receptor GGN primer has the sequence set forth in SEQ ID NO:11 and the second androgen receptor GGN primer has the sequence set forth in SEQ ID NO:12.

40. The method of claim 32, wherein the AIB1 PCR product is produced using a first AIB1 primer that selectively hybridizes with the sequence set forth in SEQ ID NO:3 and a second AIB1 primer that selectively hybridizes with the sequence set forth in SEQ ID NO:4. and wherein the androgen receptor PCR product is produced using a first androgen receptor GGN primer that selectively hybridizes with the sequence set forth in SEQ ID NO:13 and a second androgen receptor GGN primer that selectively hybridizes with the sequence set forth in SEQ ID NO:14.

41. The method of claim 40, wherein the first AIB1 primer has the sequence set forth in SEQ ID NO:1 and the second AIB1 primer has the sequence set forth in SEQ ID NO:2 and wherein the first androgen receptor GGN primer has the sequence set forth in SEQ ID NO:11 and the second androgen receptor GGN primer has the sequence set forth in SEQ ID NO:12.

42. The method of claim 30, wherein more than 29 contiguous CAG or CAA repeats in at least one allele of the AIB1 gene of the person and more than 23 contiguous GGN repeats in the androgen receptor gene of the person indicates an increased risk of prostate cancer in the subject.

43. The method of claim 30, wherein more than 29 contiguous CAG or CAA repeats in at least one allele of the AIB1 gene of the person and less than 23 contiguous GGN repeats in the androgen receptor gene of the person indicates an increased risk of prostate cancer in the subject.

44. The method of claim 30, wherein less than 29 contiguous CAG or CAA repeats in at least one allele of the AIB1 gene of the person and more than 23 contiguous GGN repeats in the androgen receptor gene of the person indicates an increased risk of prostate cancer in the subject.

45. The method of claim 30, wherein less than 29 contiguous CAG or CAA repeats in at least one allele of the AIB1 gene of the person and less than 23 contiguous GGN repeats in the androgen receptor gene of the person indicates an increased risk of prostate cancer in the subject.

46. The method of claim 30, wherein more or less than 29 contiguous CAG or CAA repeats in both alleles of the AIB1 gene of the person and more than 23 contiguous GGN repeats in the androgen receptor gene of the person indicates an increased risk of prostate cancer in the subject.

47. The method of claim 30, wherein more or less than 29 contiguous CAG or CAA repeats in both alleles of the AIB1 gene of the person and less than 23 contiguous GGN repeats in the androgen receptor gene of the person indicates an increased risk of prostate cancer in the subject.

48. The method of claim 30, wherein more than 29 contiguous CAG or CAA repeats in both alleles of the AIB1 gene of the person and more than 23 contiguous GGN repeats in the androgen receptor gene of the person indicates an increased risk of prostate cancer in the subject.

49. The method of claim 30, wherein less than 29 contiguous CAG or CAA repeats in both alleles of the AIB1 gene of the person and less than 23 contiguous GGN repeats in the androgen receptor gene of the person indicates an increased risk of prostate cancer in the subject.

50. A method for assessing the risk of prostate cancer in a human subject comprising determining the length of the contiguous CAG or CAA repeats in an AIB1 gene of the subject and assessing whether the length of the CAG repeats is less than, equal to, or greater than 29 repeats, a length less than or greater than 29 repeats indicating an increased risk of prostate cancer in the subject.

51. A method for assessing the risk of prostate cancer in a human subject comprising determining the length of the contiguous CAG or CAA repeats in an AIB1 gene of the subject and assessing whether the length of the CAG or CAA repeats is less than, equal to, or greater than 29 repeats, and determining the length of the contiguous CAG or CAA repeats in the androgen receptor gene of the subject and assessing whether the length of the CAG or CAA repeats is less than, equal to, or greater than 23 repeats, a length less than or greater than 29 repeats in the AIB1 allele and less than or greater than 23 repeats in the androgen receptor gene indicating an increased risk of prostate cancer in the subject.

52. A method for assessing the risk of prostate cancer in a human subject comprising determining the length of the contiguous CAG or CAA repeats in an AIB1 gene of the subject and assessing whether the length of the CAG or CAA repeats is less than, equal to, or greater than 29 repeats, and determining the length of the contiguous GGN repeats in the androgen receptor gene of the subject and assessing whether the length of the GGN repeats is less than, equal to, or greater than 23 repeats, a length less than or greater than 29 repeats in the AIB1 allele and less than or greater than 23 repeats in the androgen receptor gene indicating an increased risk of prostate cancer in the subject, wherein N is either T, G, or C.

53. A kit for assessing a subject's risk for acquiring prostate cancer, comprising the oligonucleotides set forth in SEQ ID Nos: 1 and 2.

54. A composition comprising the primers having the sequence set forth in SEQ ID Nos: 1 and 2.


SEQUENCE LISTING (21 sequences)

SEQ ID NO: 1 - DNA, 22 bp, Artificial Sequence
  Description of Artificial Sequence/note = synthetic construct
tcatcacctccgacaacagagg

SEQ ID NO: 2 - DNA, 22 bp, Artificial Sequence
  Description of Artificial Sequence/note = synthetic construct
tatggaaactgttgcggaggag

SEQ ID NO: 3 - DNA, 22 bp, Artificial Sequence
  Description of Artificial Sequence/note = synthetic construct
cctctgttgtcggaggtgatga

SEQ ID NO: 4 - DNA, 22 bp, Artificial Sequence
  Description of Artificial Sequence/note = synthetic construct
ctcctccgcaacagtttccata

SEQ ID NO: 5 - DNA, 48 bp, Artificial Sequence
  misc_feature 1, 2, 3, 7, 8, 9: Sequence can be repeated one or more times
cagcaacagcaacagcaacagcaacagcaacagcagcaacagcagcaa

SEQ ID NO: 6 - DNA, 87 bp, Artificial Sequence
  Description of Artificial Sequence/note = synthetic construct
cagcagcagcagcagcagcaacagcagcagcagcagcagcagcagcagcaacagcaacagcaacagcaacagcagcaacagcagcaa

SEQ ID NO: 7 - DNA, 21 bp, Artificial Sequence
  Description of Artificial Sequence/note = synthetic construct
gctctgggacgcaacctctct

SEQ ID NO: 8 - DNA, 21 bp, Artificial Sequence
  Description of Artificial Sequence/note = synthetic construct
gcagcgactaccgcatcatca

SEQ ID NO: 9 - DNA, 21 bp, Artificial Sequence
  Description of Artificial Sequence/note = synthetic construct
agagaggttgcgtcccagagc

SEQ ID NO: 10 - DNA, 21 bp, Artificial Sequence
  Description of Artificial Sequence/note = synthetic construct
tgatgatgcggtagtcgctgc

SEQ ID NO: 11 - DNA, 24 bp, Artificial Sequence
  Description of Artificial Sequence/note = synthetic construct
accctcagccgccgcttcctcatc

SEQ ID NO: 12 - DNA, 24 bp, Artificial Sequence
  Description of Artificial Sequence/note = synthetic construct
ctgggatagggcactctgctcaac

SEQ ID NO: 13 - DNA, 24 bp, Artificial Sequence
  Description of Artificial Sequence/note = synthetic construct
gatgaggaagcggcggctgagggt

SEQ ID NO: 14 - DNA, 24 bp, Artificial Sequence
  Description of Artificial Sequence/note = synthetic construct
gttgagcagagtgccctatcccag

SEQ ID NO: 15 - DNA, 24 bp, Artificial Sequence
  Description of Artificial Sequence/note = synthetic construct
cggggtaagggaagtaggtggaag

SEQ ID NO: 16 - DNA, 24 bp, Artificial Sequence
  Description of Artificial Sequence/note = synthetic construct
ctctacgatgggcttggggagaac

SEQ ID NO: 17 - DNA, 21 bp, Artificial Sequence
  misc_feature 21: Sequence can be repeated one or more times
ggtggtggtgggggtggtggc

SEQ ID NO: 18 - DNA, 6832 bp, Artificial Sequence
  Description of Artificial Sequence/note = synthetic construct
cggcagcggctgcggcttagtcggtggcggccggcggcggctgcgggctgagcggcgagtttccgatttaaagctgagctgcgaggaaaatggcggcgggaggatcaaaatacttgctggatggtggactcagagaccaataaaaataaactgcttgaacatcctttgactggttagccagttgctgatgtatattcaagatgagtggattaggagaaaacttggatccactggccagtgattcacgaaaacgcaaattgccatgtgatactccaggacaaggtcttacctgcagtggtgaaaaacggagacgggagcaggaaagtaaatatattgaagaattggctgagctgatatctgccaatcttagtgatattgacaatttcaatgtcaaaccagataaatgtgcgattttaaaggaaacagtaagacagatacgtcaaataaaagagcaaggaaaaactatttccaatgatgatgatgttcaaaaagccgatgtatcttctacagggcagggagttattgataaagactccttaggaccgcttttacttcaggcattggatggtttcctatttgtggtgaatcgagacggaaacattgtatttgtatcagaaaatgtcacacaatacctgcaatataagcaagaggacctggttaacacaagtgtttacaatatcttacatgaagaagacagaaaggattttcttaagaatttaccaaaatctacagttaatggagtttcctggacaaatgagacccaaagacaaaaaagccatacatttaattgccgtatgttgatgaaaacaccacatgatattctggaagacataaacgccagtcctgaaatgcgccagagatatgaaacaatgcagtgctttgccctgtctcagccacgagctatgatggaggaaggggaagatttgcaatcttgtatgatctgtgtggcacgccgcattactacaggagaaagaacatttccatcaaaccctgagagctttattaccagacatgatctttcaggaaaggttgtcaatatagatacaaattcactgagatcctccatgaggcctggctttgaagatataatccgaaggtgtattcagagattttttagtctaaatgatgggcagtcatggtcccagaaacgtcactatcaagaagcttatcttaatggccatgcagaaaccccagtatatcgattctcgttggctgatggaactatagtgactgcacagacaaaaagcaaactcttccgaaatcctgtaacaaatgatcgacatggctttgtctcaacccacttccttcagagagaacagaatggatatagaccaaacccaaatcctgttggacaagggattagaccacctatggctggatgcaacagttcggtaggcggcatgagtatgtcgccaaaccaaggcttacagatgccgagcagcagggcctatggcttggcagaccctagcaccacagggcagatgagtggagctaggtatgggggttccagtaacatagcttcattgacccctgggccaggcatgcaatcaccatcttcctaccagaacaacaactatgggctcaacatgagtagccccccacatgggagtcctggtcttgccccaaaccagcagaatatcatgatttctcctcgtaatcgtgggagtccaaagatagcctcacatcagttttctcctgttgcaggtgtgcactctcccatggcatcttctggcaatactgggaaccacagcttttccagcagctctctcagtgccctgcaagccatcagtgaaggtgtggggacttcccttttatctactctgtcatcaccaggccccaaattggataactctcccaatatgaatattacccaaccaagtaaagtaagcaatcaggattccaagagtcctctgggcttttattgcgaccaaaatccagtggagagttcaatgtgtcagtcaaatagcagagatcacctcagtgacaaagaaagtaaggagagcagtgttgagggggcagagaatcaaaggggtcctttggaaagcaaaggtcataaaaaattactgcagttacttacctgttcttctgatgaccggggtcattcctccttgaccaactcccccctagattcaagttgtaaagaatcttctgttagtgtcaccagcccctctggagtctcctcctctacatctggaggagtatcctctacatccaatatgcatgggtcactgttacaagagaagcaccggattttgcacaagttgctgcagaatgggaattcaccagctgaggtagccaagattactgcagaagccactgggaaagacaccagcagtataacttcttgtggggacggaaatgttgtcaagcaggagcagctaagtcctaagaagaaggagaataatgcacttcttagatacctgctggacagggatgatcctagtgatgcactctctaaagaactacagccccaagtggaaggagtggataataaaatgagtcagtgcaccagctccaccattcctagctcaagtcaagagaaagaccctaaaattaagacagagacaagtgaagagggatctggagacttggataatctagatgctattcttggtgatctgactagttctgacttttacaataattccatatcctcaaatggtagtcatctggggactaagcaacaggtgtttcaaggaactaattctctgggtttgaaaagttcacagtctgtgcagtctattcgtcctccatataaccgagcagtgtctctggatagccctgtttctgttggctcaagtcctccagtaaaaaatatcagtgctttccccatgttaccaaagcaacccatgttgggtgggaatccaagaatgatggatagtcaggaaaattatggctcaagtatgggtgggccaaaccgaaatgtgactgtgactcagactccttcctcaggagactggggcttaccaaactcaaaggccggcagaatggaacctatgaattcaaactccatgggaagaccaggaggagattataatacttctttacccagacctgcactgggtggctctattcccacattgcctcttcggtctaatagcataccaggtgcgagaccagtattgcaacagcagcagcagatgcttcaaatgaggcctggtgaaatccccatgggaatgggggctaatccctatggccaagcagcagcatctaaccaactgggttcctggcccgatggcatgttgtccatggaacaagtttctcatggcactcaaaataggcctcttcttaggaattccctggatgatcttgttgggccaccttccaacctggaaggccagagtgacgaaagagcattattggaccagctgcacactcttctcagcaacacagatgccacaggcctggaagaaattgacagagctttgggcattcctgaacttgtcaatcagggacaggcattagagcccaaacaggatgctttccaaggccaagaagcagcagtaatgatggatcagaaggcaggattatatggacagacatacccagcacaggggcctccaatgcaaggaggctttcatcttcagggacaatcaccatcttttaactctatgatgaatcagatgaaccagcaaggcaattttcctctccaaggaatgcacccacgagccaacatcatgagaccccggacaaacacccccaagcaacttagaatgcagcttcagcagaggctgcagggccagcagtttttgaatcagagccgacaggcacttgaattgaaaatggaaaaccctactgctggtggtgctgcggtgatgaggcctatgatgcagccccagcagggttttcttaatgctcaaatggtcgcccaacgcagcagagagctgctaagtcatcacttccgacaacagagggtggctatgatgatgcagcagcagcagcagcagcaacagcagcagcagcagcagcagcagcagcaacagcaacagcaacagcaacagcagcaacagcagcaaacccaggccttcagcccacctcctaatgtgactgcttcccccagcatggatgggcttttggcaggacccacaatgccacaagctcctccgcaacagtttccatatcaaccaaattatggaatgggacaacaaccagatccagcctttggtcgagtgtctagtcctcccaatgcaatgatgtcgtcaagaatgggtccctcccagaatcccatgatgcaacacccgcaggctgcatccatctatcagtcctcagaaatgaagggctggccatcaggaaatttggccaggaacagctccttttcccagcagcagtttgcccaccaggggaatcctgcagtgtatagtatggtgcacatgaatggcagcagtggtcacatgggacagatgaacatgaaccccatgcccatgtctggcatgcctatgggtcctgatcagaatactgctgacatctctgcaccaggacctcttaaggaaaccactgtacaaatgacactgcactaggattattgggaaggaatcattgttccaggcatccatcttggaagaaaggaccagctttgagctccatcaagggtattttaagtgatgtcatttgagcaggactggattttaagccgaagggcaatatctacgtgtttttcccccctccttctgctgtgtatcatggtgttcaaaacagaaatgttttttggcattccacctcctagggatataattctggagacatggagtgttactgatcataaaacttttgtgtcacttttttctgccttgctagccaaaatctcttaaatacacgtaggtgggccagagaacattggaagaatcaagagagattagaatatctggtttctctagttgcagtattggacaaagagcatagtcccagccttcaggtgtagtagttctgtgttgaccctttgtccagtggaattggtgattctgaattgtcctttactaatggtgttgagttgctctgtccctattatttgccctaggctttctcctaatgaaggttttcatttgccattcatgtcctgtaatacttcacctccaggaactgtcatggatgtccaaatggctttgcagaaaggaaatgagatgacagtatttaatcgcagcagtagcaaacttttcacatgctaatgtgcagctgagtgcactttatttaaaaagaatggataaatgcaatattcttgaggtcttgagggaatagtgaaacacattcctggtttttgcctacacttacgtgttagacaagaactatgatttttttttttaaagtactggtgtcaccctttgcctatatggtagagcaataatgctttttaaaaataaacttctgaaaacccaaggccaggtactgcattctgaatcagaatctcgcagtgtttctgtgaatagatttttttgtaaatatgacctttaagatattgtattatgtaaaatatgtatatacctttttttgtaggtcacaacaactcatttttacagagtttgtgaagctaaatatttaacattgttgatttcagtaagctgtgtggtgaggctaccagtggaagagacatcccttgacttttgtggcctgggggaggggtagtgctccacagcttttccttccccaccccccagccttagatgcctcgctcttttcaatctcttaatctaaatgctttttaaagagattatttgtttagatgtaggcattttaattttttaaaaattcctctaccagaactaagcactttgttaatttggggggaaagaatagatatggggaaataaacttaaaaaaaaatcaggaatttaaaaaaacgagcaatttgaagagaatcttttggattttaagcagtccgaaataatagcaattcatgggctgtgtgtgtgtgtgtatgtgtgtgtgtgtgtgtgtatgtttaattatgttaccttttcatcccctttaggagcgttttcagattttggttgctaagacctgaatcccatattgagatctcgagtagaatccttggtgtggtttctggtgtctgctcagctgtcccctcattctactaatgtgatgctttcattatgtccctgtggattagaatagtgtcagttatttcttaagtaactcagtacccagaacagccagttttactgtgattcagagccacagtctaactgagcaccttttaaacccctccctcttctgccccctaccacttttctgctgttgcctctctttgacacctgttttagtcagttgggaggaagggaaaaatcaagtttaattccctttatctgggttaattcatttggttcaaatagttgacggaattgggtttctgaatgtctgtgaatttcagaggtctctgctagccttggtatcattttctagcaataactgagagccagttaattttaagaatttcacacatttagccaatctttctagatgtctctgaaggtaagatcatttaatatctttgatatgcttacgagtaagtgaatcctgattatttccagacccaccaccagagtggatcttattttcaaagcagtatagacaattatgagtttgccctctttcccctaccaagttcaaaatatatctaagaaagattgtaaatccgaaaacttccattgtagtggcctgtgcttttcagatagtatactctcctgtttggagacagaggaagaaccaggtcagtctgtctctttttcagctcaattgtatctgacccttctttaagttatgtgtgtggggagaaatagaatggtgctcttatctttcttgactttaaaaaaattattaaaaacaaaaaaaaaataaa

SEQ ID NO: 19 - protein, 1438 aa, Artificial Sequence
  Description of Artificial Sequence/note = synthetic construct
MSGLGENLDPLASDSRKRKLPCDTPGQGLTCSGEKRRREQESKYIEELAELISANLSDIDNFNVKPDKCAILKETVRQIRQIKEQGKTISNDDDVQKADVSSTGQGVIDKDSLGPLLLQALDGFLFVVNRDGNIVFVSENVTQYLQYKQEDLVNTSVYNILHEEDRKDFLKNLPKSTVNGVSWTNETQRQKSHTFNCRMLMKTPHDILEDINASPEMRQRYETMQCFALSQPRAMMEEGEDLQSCMICVARRITTGERTFPSNPESFITRHDLSGKVVNIDTNSLRSSMRPGFEDIIRRCIQRFFSLNDGQSWSQKRHYQEAYLNGHAETPVYRFSLADGTIVTAQTKSKLFRNPVTNDRHGFVSTHFLQREQNGYRPNPNPVGQGIRPPMAGCNSSVGGMSMSPNQGLQMPSSRAYGLADPSTTGQMSGARYGGSSNIASLTPGPGMQSPSSYQNNNYGLNMSSPPHGSPGLAPNQQNIMISPRNRGSPKIASHQFSPVAGVHSPMASSGNTGNHSFSSSSLSALQAISEGVGTSLLSTLSSPGPKLDNSPNMNITQPSKVSNQDSKSPLGFYCDQNPVESSMCQSNSRDHLSDKESKESSVEGAENQRGPLESKGHKKLLQLLTCSSDDRGHSSLTNSPLDSSCKESSVSVTSPSGVSSSTSGGVSSTSNMHGSLLQEKHRILHKLLQNGNSPAEVAKITAEATGKDTSSITSCGDGNVVKQEQLSPKKKENNALLRYLLDRDDPSDALSKELQPQVEGVDNKMSQCTSSTIPSSSQEKDPKIKTETSEEGSGDLDNLDAILGDLTSSDFYNNSISSNGSHLGTKQQVFQGTNSLGLKSSQSVQSIRPPYNRAVSLDSPVSVGSSPPVKNISAFPMLPKQPMLGGNPRMMDSQENYGSSMGGPNRNVTVTQTPSSGDWGLPNSKAGRMEPMNSNSMGRPGGDYNTSLPRPALGGSIPTLPLRSNSIPGARPVLQQQQQMLQMRPGEIPMGMGANPYGQAAASNQLGSWPDGMLSMEQVSHGTQNRPLLRNSLDDLVGPPSNLEGQSDERALLDQLHTLLSNTDATGLEEIDRALGIPELVNQGQALEPKQDAFQGQEAAVMMDQKAGLYGQTYPAQGPPMQGGFHLQGQSPSFNSMMNQMNQQGNFPLQGMHPRANIMRPRTNTPKQLRMQLQQRLQGQQFLNQSRQALELKMENPTAGGAAVMRPMMQPQQGFLNAQMVAQRSRELLSHHFRQQRVAMMMQQQQQQQQQQQQQQQQQQQQQQQQQQQQQTQAFSPPPNVTASPSMDGLLAGPTMPQAPPQQFPYQPNYGMGQQPDPAFGRVSSPPNAMMSSRMGPSQNPMMQHPQAASIYQSSEMKGWPSGNLARNSSFSQQQFAHQGNPAVYSMVHMNGSSGHMGQMNMNPMPMSGMPMGPDQNTADISAPGPLKETTVQMTLH

SEQ ID NO: 20 - DNA, 4321 bp, Artificial Sequence
  Description of Artificial Sequence/note = synthetic construct
cgagatcccggggagccagcttgctgggagagcgggacggtccggagcaagcccacaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttagaattccggcggagagaaccctctgttttcccccactctctctccacctcctcctgccttccccaccccgagtgcggagcagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaaacaagcaaacaaaaaaaaaa

SEQ ID NO: 21 - protein, 919 aa, Artificial Sequence
  Description of Artificial Sequence/note = synthetic construct
MEVQLGLGRVYPRPPSKTYRGAFQNLFQSVREVIQNPGPRHPEAASAAPPGASLLLLQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ